(19) United States
(12) Patent Application Publication
(10) Pub. No.: US 2019/0269755 A1
(43) Pub. Date: Sep. 5, 2019

(54) OPTOGENETIC VISUAL RESTORATION USING CHRIMSON

(71) Applicants: Gensight Biologics SA, Paris (FR); UNIVERSITE PIERRE ET MARIE CURIE (PARIS 6), Paris (FR); CENTRE NATIONAL DE LA RECHERCHE SCIENTIFIQUE, Paris (FR); INSITITUT NATIONAL DE LA SANTE DE LA RECHERCHE MEDICALE, Paris (FR)

(72) Inventors: Didier PRUNEAU, Paques (FR); Anne DOUAR, Paris (FR); Deniz DALKARA, Alfortville (FR); Jens DUEBEL, Paris (FR); Romain CAPLETTE, Paris (FR); Gregory GAUVAIN, Paris (FR); Melissa DESROSIERS, Paris (FR); Jose SAHEL, Paris (FR); Serge PICAUD, Avon (FR)

(21) Appl. No.: 16/097,204

(22) PCT Filed: Apr. 28, 2017
(86) PCT No.: PCT/IB2017/000663

§ 371 Date: Feb. 11, 2019

Related U.S. Application Data
(60) Provisional application No. 62/329,692, filed on Apr. 29, 2016.

(51) Int. Cl. A61K 38/17; A61K 38/16; A61K 35/761; A61K 9/00; A61P 27/02

(52) U.S. Cl. CPC A61K 38/1709; A61K 38/168; A61K 38/1767; A61K 48/00; A61K 9/0048; A61K 9/0019; A61P 27/02; A61K 35/761

(57) ABSTRACT

Disclosed are, among other methods, methods for reactivating retinal ganglion cells in mammals by administering an effective amount of channelrhodopsins (such as ChrimsonR), or an effective amount of such channelrhodopsins (such as ChrimsonR) fused to a fluorescent protein, in the form of protein or nucleic acids, and compositions thereof. The methods may include a light stimuli level inducing RGCs response that is below radiation safety limit. The methods may include delivery by an adenoassociated virus vector. The methods may include use of a CAG promoter. The methods may result in a long term expression of an effective amount of the channelrhodopsins (such as ChrimsonR protein).

### CROSS-REFERENCE TO RELATED APPLICATIONS

This application is the United States national stage entry under 35 U.S.C. § 371 of International Application No. PCT/IB2017/000663, filed on Apr. 28, 2017, which claims the benefit of priority of U.S. Provisional Application No. 62/329,692, filed on Apr. 29, 2016. The contents of which these applications are each incorporated herein by reference in their entirety.

### SEQUENCE LISTING

The instant application contains a Sequence Listing which has been submitted electronically in ASCII format and is hereby incorporated by reference in its entirety. Said ASCII copy, created on Apr. 28, 2017, is named 12295_0006-00304.txt and is 31 bytes in size.

### FIELD

The present disclosure provides, among other things, compositions and methods for altering conductance across membranes, cell activity, and cell function, and relates to the use of exogenous light-activated ion channels in cells and subjects. More particularly, an aspect of an embodiment of the present invention relates to a method for reactivating retinal ganglion cells (RGCs) in mammals comprising administering to a mammal an effective amount of a Chrimson polypeptide. In some embodiments, the method may include a light stimuli level inducing RGCs response below the radiation safety limit. In some embodiments, the Chrimson polypeptide is fused to a fluorescent protein. In some embodiments the fluorescent protein is tdTomato (tdT) or green fluorescent protein (GFP).

### BACKGROUND OF THE INVENTION

The retina is composed of photoreceptors, which are highly specialized neurons that are responsible for photosensitivity of the retina by phototransduction, i.e. the conversion of light into electrical and chemical signals that propagate a cascade of events within the visual system, ultimately generating a representation of world. In the vertebrate retina, phototransduction is initiated by activation of light-sensitive receptor protein, rhodopsin.

Photoreceptor loss or degeneration, such as in case of retinitis pigmentosa (RP) or macular deneneration (MD), severely compromises, if not completely inhibits, phototransduction of visual information within the retina. Loss of photoreceptor cells and/or loss of a photoreceptor cell function are the primary causes of diminished visual acuity, diminished light sensitivity, and blindness.

Several therapeutic approaches dedicated to retinal degenerative diseases are currently in development, including gene therapy, stem cell therapy, optogenetics, and retinal prostheses (Scholl et al., 2016, Science Translational Medicine, 8 (368), 368rv6).

For example it has been proposed to restore photosensitivity of the retina of a subject by controlling activity of defined populations of neurons without affecting other neurons in the brain by gene- and neuroengineering technology termed optogenetics. In contrast to traditional gene therapy that attempts to replace or repair a defective gene or bypass the genetic defect through correction of the protein deficiency or dysfunction, optogenetic approaches to therapy can be used to endow normally non-photosensitive cells in the retina with the ability to respond to light, thus restoring useful vision to the patient. Unlike retinal chip implants that provide extracellular electrical stimulation to bipolar or ganglion cells, optogenetics-based therapies stimulate the cells from inside the cell.

Optogenetics (Deisseroth. Nat Methods 8 (1): 26-9, 2011) refers to the combination of genetics and optics to control well-defined events within specific cells of living tissue. Optogenetics involves the introduction into cells of light-activated channels that allow manipulation of neural activity with millisecond precision while maintaining cell-type resolution through the use of specific targeting mechanisms. It includes the discovery and insertion into cells of genes that confer light responsiveness; it also includes the associated technologies for delivering light deep into organisms as complex as mammals, for targeting light-sensitivity to cells of interest, and for assessing specific readouts, or effects, of this optical control.

For example WO2007024391, WO2008022772 or WO2009127705 describe the use of opsin genes derived from plants and microbial organisms (e.g. archaebacteria, bacteria, and fungi) encoding light-activated ion channels and pumps (e.g. channelrhodopsin-2 [ChR2]; halorhodopsin [NpHR]), engineered for expression in mammalian neurons and which can be genetically targeted into specific neural populations using viral vectors. When exposed to light with appropriate wavelength, action potentials can be triggered in opsin-expressing neurons conferring thereby light sensitivity to these cells.

In recent years, a number of channelrhodopsins have been engineered for neuroscientific applications, derived from four channelrhodopsin genes from Chlamydomonas reinhardtii or Volvox carteri. However, those natural channelrhodopsins have only blue-green (430-550 nm) spectral peaks, and engineered red-shifted channelrhodopsins such as C1V1 and ReaChR have peak wavelength sensitivity in the green (˜545 nm) (Mattis et al., Nature Methods, 2011 Dec. 18; 9(2):159-72; Lin et al., Nature Neuroscience, 2013 Oct. 16 (10):1499-508).

In 2014, Klapoetke et al., Nat Methods, 11 (3), 338-346 have therefore sought to overcome these limitations through exploring natural channelrhodopsin genetic diversity, aiming to discover new opsins possessing unique features not found in previously described channelrhodopsins. WO2013071231 thus discloses new channelrhodopsins, Chronos and Chrimson, which have different activation spectra from one another and from the state of the art (e.g., ChR2/VChR1), and allow multiple and distinct wavelengths of light to be used to depolarize different sets of cells in the same tissue, by expressing channels with different activation spectra genetically expressed in different cells, and then illuminating the tissue with different colors of light. More particularly, Chrimson is 45 nm red-shifted relative to any previous channelrhodopsin; this could be important for situations where red light would be preferred, as red light is more weakly scattered by tissue and absorbed less by blood than the blue to green wavelengths required by other channelrhodopsin variants.

Opsins are often fused to fluorescent proteins to facilitate visualization in opsin-expressing cells and thus to monitor their intracellular localization. It has further being shown that some types of fluorescent protein used can in certain conditions modulate opsin cellular localisation. For example, Arrenberg et al. (2009, PNAS, 106 (42), 17968-73) have observed that fusion proteins containing the identical opsin but different fluorescent tags (i.e. red fluorescent protein mCherry or yellow fluorescent protein YFP) are sometimes distributed in different cellular compartments.

However this observation was not confirmed with tdTomato fluorescent tag, as no apparent difference in expression level or membrane localization was seen in transgenic animals expressing channelrhodopsin-2 fused to tdTomato (Madisen et al. 2012, Nat Neurosci., 15 (5): 793-802). Moreover, no improvements have been reported to date on the activity of the opsins that are associated with this change in localization or expression level of the fusion protein.

### SUMMARY OF THE INVENTION

In one embodiment, this disclosure shows that the Chrimson protein, and more particularly one special mutant thereof called Chrimson R (ChrR), fused to a tdTomato (tdT) fluorescent protein or green fluorescent protein (GFP) is more effective in responding to light stimuli compared to Chrimson protein alone. In some embodiments of the method, the fluorescent protein increases the expression level, more particularly the protein level at the plasma membrane, of the fused Chrimson protein for a given number of cells compared with the expression level of the Chrimson protein alone/unfused. In some other embodiments of the method the fluorescent protein increases the cellular trafficking of the fused Chrimson to the plasma membrane compared with the cellular trafficking of the Chrimson protein alone/unfused. In some embodiments of the method, the expression level and/or cellular trafficking of the fused Chrimson protein is increased through enhanced solubility, trafficking, and/or protein conformation of the Chrimson protein.

In an aspect, the present disclosure encompasses a polynucleotide sequence encoding Chrimson protein and a fluorescent protein.

In another aspect, the present disclosure encompasses a polynucleotide sequence encoding Chrimson protein fused to a fluorescent protein.

In another aspect, the present disclosure encompasses a composition comprising a vector. The vector comprises a polynucleotide sequence encoding a polypeptide, the polypeptide comprising at least one Chrimson protein and a fluorescent protein.

In still another aspect, the present disclosure encompasses a composition comprising a vector comprising a polynucleotide sequence encoding a polypeptide, the polypeptide comprising Chrimson protein fused to a fluorescent protein.

In still yet another aspect, the present disclosure encompasses a method of treating or preventing neuron mediated disorders in a subject wherein the method comprises administering to the cell (i.e., the neuron) a composition comprising a vector. The vector comprises a polynucleotide sequence encoding a polypeptide, the polypeptide comprising at least one Chrimson protein and a fluorescent protein. Preferably, the vector of the administered composition comprises a polynucleotide sequence encoding a polypeptide, the polypeptide comprising Chrimson protein fused to a fluorescent protein.

In still yet another aspect, the present disclosure encompasses a method of restoring sensitivity to light in an inner retinal cell. The method comprises administering to the cell a composition comprising a vector. The vector comprises a polynucleotide sequence encoding a polypeptide, the polypeptide comprising at least one Chrimson protein and a fluorescent protein. Preferably, the vector of the administered composition comprises a polynucleotide sequence encoding a polypeptide, the polypeptide comprising Chrimson protein fused to a fluorescent protein.

In a different aspect, the present disclosure encompasses a method of restoring vision to a subject. The method comprises identifying a subject with loss of vision due to a deficit in light perception or sensitivity; administering a composition comprising a vector to the eye?, the vector comprising a polynucleotide sequence encoding a polypeptide, the polypeptide comprising at least one Chrimson protein and a fluorescent protein; activating the polypeptide with light; and measuring light sensitivity in the subject, wherein increased light sensitivity indicates vision restoration.

In another aspect, the present disclosure encompasses a method of restoring vision to a subject wherein the method comprises identifying a subject with loss of vision due to a deficit in light perception or sensitivity; administering a composition comprising a vector to the eye, the vector comprising a polynucleotide sequence encoding a polypeptide, the polypeptide comprising at least one Chrimson protein fused to a fluorescent protein; activating the polypeptide with light; and measuring light sensitivity in the subject, wherein increased light sensitivity indicates vision restoration.

In other aspects, the present disclosure encompasses a method of treating or preventing retinal degeneration in a subject. The method comprises identifying a subject with retinal degeneration due to loss of photoreceptor function; administering a composition comprising a vector to the eye, the vector comprising a polynucleotide sequence encoding a polypeptide, the polypeptide comprising at least one Chrimson protein and a fluorescent protein; and measuring light-sensitivity in the subject, wherein increased sensitivity to light indicates treatment of retinal degeneration.

In still another aspects, the present disclosure encompasses a method of treating or preventing retinal degeneration in a subject wherein the method comprises identifying a subject with retinal degeneration due to loss of photoreceptor function; administering a composition comprising a vector, the vector comprising a polynucleotide sequence encoding a polypeptide, the polypeptide comprising at least one Chrimson protein fused to a fluorescent protein; and measuring light-sensitivity in the subject, wherein increased sensitivity to light indicates treatment of retinal degeneration.

In certain aspects, the present disclosure encompasses a method of restoring photoreceptor function in a human eye. The method comprises administering an effective amount of composition comprising a vector, the vector comprising a polynucleotide sequence encoding a polypeptide, the polypeptide comprising at least one Chrimson protein and a fluorescent protein.

In another aspect, the present disclosure encompasses a method of restoring photoreceptor function in a human eye said method comprises administering an effective amount of composition comprising a vector, the vector comprising a polynucleotide sequence encoding a polypeptide, the polypeptide comprising at least one Chrimson protein fused to a fluorescent protein.

In yet other aspects, the present disclosure encompasses a method of depolarizing an electrically active cell. The method comprises administering to the cell a composition comprising a vector, the vector comprising a polynucleotide sequence encoding a polypeptide, the polypeptide comprising at least one Chrimson protein and a fluorescent protein.

In yet another aspect, the present disclosure encompasses a method of depolarizing an electrically active cell said method comprises administering to the cell a composition comprising a vector, the vector comprising a polynucleotide sequence encoding a polypeptide, the polypeptide comprising at least one Chrimson protein fused to a fluorescent protein.

In some embodiments of the method of the present disclosure, the vector is an adenoassociated virus (AAV) vector. In some embodiments of the method, the vector is an AAV2.7m8 vector or an AAV2 vector. In some embodiments the method further comprises the use of a CAG promoter.

In some embodiment the vector is administered by injection, preferably is injected intravitreally.

In some embodiments of the method, the effective amount of the Chrimson protein is expressed for a long term. In some embodiments of the method, the expression of the Chrimson protein is persistent after at least 11 months post injection. In some embodiments of the method, the expression of the Chrimson protein is persistent after at least 2 months post injection.

In some embodiments of the method, the subject is a mammal. In some embodiments, the subject is a human. In some embodiments, the mammal is a mouse. In some embodiments of the method, the mouse is rd1. In some embodiments of the method the mammal is a rat. In some embodiments of the method, the rat is P23H. In some embodiments of the method, the mammal is a human or non-human primate. In some embodiments of the method, the non-human primate is a cynomolgus macaque.

The following disclosure also provides the following additional embodiments:

Embodiment 1 provides a method for reactivating retinal ganglion cells (RGCs) in mammals comprising administering to a mammal a vector expressing an effective amount of Chrimson protein fused to a fluorescent protein.

Embodiment 2 provides a method of treating or preventing neuron mediated disorders in a subject wherein the method comprises administering to a neuron a composition comprising a vector expressing an effective amount of Chrimson protein fused to a fluorescent protein.

Embodiment 3 provides a method of restoring sensitivity to light in an inner retinal cell wherein the method comprises administering to an inner retinal cell a composition comprising a vector expressing an effective amount of Chrimson protein fused to a fluorescent protein.

Embodiment 4 provides a method of restoring vision to a subject wherein the method comprises administering to the subject a composition comprising a vector expressing an effective amount of Chrimson protein fused to a fluorescent protein.

Embodiment 5 provides a method of restoring vision to a subject wherein the method comprises identifying a subject with loss of vision due to a deficit in light perception or sensitivity and administering to the subject a composition comprising a vector expressing an effective amount of Chrimson protein fused to a fluorescent protein.

Embodiment 6 provides a method of treating or preventing retinal degeneration in a subject wherein the method comprises identifying a subject with retinal degeneration due to loss of photoreceptor function and administering to the subject a composition comprising a vector expressing an effective amount of Chrimson protein fused to a fluorescent protein.

Embodiment 7 provides a method of restoring photoreceptor function in a human eye wherein the method comprises identifying a subject with loss of vision due to a deficit in light perception or sensitivity and administering to the subject a composition comprising a vector expressing an effective amount of Chrimson protein fused to a fluorescent protein.

Embodiment 8 provides a method of depolarizing an electrically active cell wherein the method comprises administering to the cell a composition comprising a vector expressing an effective amount of Chrimson protein fused to a fluorescent protein.

Embodiment 9 provides a method according to any one of embodiments 1 through 8, wherein a light stimuli level inducing RGCs response is below radiation safety limit.

Embodiment 10 provides a method according to any one of embodiments 1 through 8, wherein the Chrimson protein is Chrimson 88 or Chrimson R.

Embodiment 11 provides a method of embodiment 10, wherein the fluorescent protein is selected from Td-Tomato (TdT) protein and green fluorescent protein (GFP).

Embodiment 12 provides a method of embodiment 11, wherein the Chrimson protein fused to the tdT protein is more effective in responding to light stimuli compared with Chrimson protein alone.

Embodiment 13 provides a method of embodiment 10, wherein the fluorescent protein increases the expression level of the fused Chrimson protein for a given number of cells compared with the expression level of the Chrimson protein alone.

Embodiment 14 provides a method of embodiment 13, wherein the expression level of the fused Chrimson protein is increased through enhanced solubility, trafficking, and/or protein conformation of the Chrimson protein.

Embodiment 15 provides a method according to any one of embodiments 1 through 8, wherein the vector is an adenoassociated virus (AAV) vector.

Embodiment 16 provides a method of embodiment 15 wherein the AAV vector is selected from AAV2 vector and AAV2.7m8 vector.

Embodiment 17 provides a method of embodiment 16, wherein the AAV vector is AAV2.7m8 vector.

Embodiment 18 provides a method according to any one of embodiments 1 through 8, wherein the vector comprises a CAG promoter.

Embodiment 19 provides a method according to any one of embodiments 1 through 8, wherein the vector is injected intravitreally.

Embodiment 20 provides a method according to any one of embodiments 1 through 8, wherein an effective amount of the Chrimson protein fused to a fluorescent protein is expressed long term.

Embodiment 21 provides a method of embodiment 20, wherein the expression of the Chrimson protein fused to a fluorescent protein is persistent after at least 2 months post administration, or at least 11 months post administration.

Embodiment 22 provides a composition comprising one or more of the vectors according to any one of embodiments 1 through 21.

Embodiment 23 provides a composition comprising one or more polynucleotides encoding one or more Chrimson proteins and one or more fluorescent proteins, fused or separately.

Embodiment 24 provides composition according to any one of claims 22 and 23, for use in one or more of the methods of any one of claims 1 through 21.

Embodiment 25 provides for the use of any one of the compositions of claims 22 and 23 to reactivate retinal ganglion cells (RGCs) in mammals, treat or prevent neuron mediated disorders in a subject, restore sensitivity to light in an inner retinal cell, treat or prevent retinal degeneration in a subject, restore photoreceptor function, and/or depolarize an electrically active cell.

### BRIEF DESCRIPTION OF THE DRAWINGS

FIG. 1: In vivo methods in rd1 mice.

FIGS. 2A through 2D: Degenerated rd1 mice retinas respond to light at a wavelight matching ChrimsonR spectral sensitivity and to duration below 10 ms. FIG. 2A—Eye fundus of a ChrR-tdT expressing rd1 mouse at 2 month post injection. FIG. 2B. TdT fluorescence of a rd1 mouse retina mounted on a MEA chip. FIG. 2C—Spectral sensitivity of ChrR expressing mice retina (n=1 retina, 188 electrodes). FIG. 2D—Added firing rate in response to stimuli of increasing duration at 590 nm at 1e¹⁷ photons. cm⁻²s⁻¹. All recordings are done in presence of a mix of L-AP4, CNQX and CCP.

FIGS. 3A through 3C: Chrimson R is more efficient when fused with tdT in rd1 mice. FIG. 3A. Comparison between retinas infected with ChrR or ChrR-tdT was more effective in responding to light stimuli. FIG. 3B. Raw data, raster plot and average PSTH (from top to bottom, respectively) of a responding RGC of a ChrR-tdT expressing retina. FIG. 3C. Intensity plot of retinas expressing ChrR (n=4 retinas, 27 cells) or ChrR-tdT (n=6 retinas, 548 cells), showing levels of activation at different stimuli intensities.

FIGS. 4A through 4G: Expression if Chrimson R in Ganglion cells. Expression of ChrR-tdT in Retinal Ganglion Cells (RGCs) of rd1 mice. Expression of ChrR-tdT after in-vivo AAV infection was largely restricted to RGCs. FIG. 4A, FIG. 4B and FIG. 4C—Projection of a confocal stack showing membrane located expression in two examples of RGCs. FIG. 4A—Image of endogeneous tdTomato, no immunological amplification. FIG. B. Image of the labelling for our custom made ChrR antibody. FIG. 4C—Overlay of both images (FIG. 4A and FIG. 4B), magenta and cyan for tdTomato and ChrR antibody, respectively. Images taken with a 40× objective. Expression of ChrR-tdT is enriched in RGCs membranes. FIG. 4D and FIG. 4E—Projections of three optic slices showing cell body of two RGCs (see inset in FIG. 4C), taken with a 60× objective. FIG. 4F and FIG. 4G—3D surface plot of fluorescence intensity for cell bodies in FIG. 4D and FIG. 4E—, respectively. Peaks, indicating highest fluorescence intensity, are concentrated at, or near, the cells membranes.

FIGS. 5A through 5D: Chrimson R long term expression. Multielectrode Array recording rd1 mice 10 months after injection. FIG. 5A—Image of a retina expressing ChrR-tdT showing that expression is persistent at 10 months post injection. FIG. 5B—example of the activity measured on one electrode, top-light stimulus in red, middle-raster plots of the same cell responses for 10 repetitions of the flash, bottom—average PTSH (bin size: SOms). FIG. 5C—Added firing rate in response to flashes of increasing intensity (n=4 retinas, 308 electrodes). FIG. 5D—Added firing rate in response to flashes of increasing durations at 590 nm at 1e¹⁷ photons.cm⁻².s⁻¹. All recordings are done in presence of a mix of L-AP4, CNQX and CPP.

FIGS. 6A through 6B: Chrimson R reactivates P23H retinas. Multielectrode Array recording on another degenerative rodent model: P23H rats. FIG. 6A—Fluorescence image of a P23H retina on the array of multielectrode, at 1 month post injection. FIG. 6B—Added firing rate in response to stimuli of increasing intensities at 590 nm at 1e¹⁷ photons.cm⁻².s⁻¹ (n=2 retinas, 91 electrodes). All records are done in presence of a mix of L-AP4, CNQX and CPP.

FIG. 7: In vivo methods in non human primate. 4 different strategies were tested for ChrR expression in non-human primates (macaca fascicularis). 2 different constructions: ChrimsonR (ChrR) or the fused protein ChrimsonR-td-Tomato (ChrR-tdT), both under the CAG promoter. 2 different viral capsids: the wild type AAV2, and the mutant AAV2-7m8 (Dalkara et al. 2013, Science Translational Medicine, 5(189):189ra76). Injections of a single viral does (5×10¹¹ vg/eye) was performed two months before MEA (512 array, MCS) or patch clamp (see poster Chaffiol et al., abstract 599-B0072) recordings. All recordings were done in presence of synaptic blockers (LAP4 50 μM and CPP 10 μM).

FIGS. 8A through 8C: Chrimson R is expressed in the peri fovea after In vivo injection of the constructs. In vivo injection of the constructs leads to expression in RGCs of the peri-foveal ring. FIG. 8A—Infrared image of a retinal explant, an asterisk indicates the depression of the foveal pit. The black dots are the electrodes of the MEA array. FIG. 8B—Fluorescent image of the same retinal piece, infected with the AAV2.7m8-ChrR-tdT construct. Expression is restricted to the peri-foveal ring. FIG. 8C—Spectral sensitivity of the retina explant displayed in FIG. 8A & FIG. 8B. Response averaged over 10 repetitions and across all responsive electrodes. Shape of the spectrum and presence of synaptic blockers indicate that ChrR in the RGCs is the source of the recorded activity.

FIGS. 9A through 9G: Identification of the test construct leading to the most efficient transduction. Transduction is evaluated as the number of responsive electrodes and the sensitivity of the light evoked response. FIG. 9A—Example of one electrode responses to light flashes at 4 different intensities. FIG. 9B—Overview of the complete set of experiments for the 4 constructs. Active electrodes: electrodes where action potentials are detected. Responding electrodes: electrodes where firing rate was increased by light stimuli. FIG. 9C—, FIG. 9D and FIG. 9E—Population responses for each responsive retina for the different constructs. Each colored line represents individual electrode responses, averaged over 10 repetitions. Each row of graphs represent responses from one retina, each column responses of different retinas to a same light stimulus (intensity on the top in photons/cm2/sec). FIG. 9F—Average added firing rate for each responsive retina at different light intensity. spontaneous firing rate is subtracted FIG. 9G—Detail of F to better visualize response threshold. All stimuli were done at 600 nm.

FIGS. 10A through 10D: Response to perifoveal RGCs stimuli of increasing duration in a retina infected with AAV2.7m8-ChR-tdT. Response of peri-foveal RGCs to stimuli of increasing duration in a retina infected with AAV2.7m8-ChrR-tdT. FIG. 10A—Responses to light stimuli of increasing duration, each line represent a single electrode spike density function average over 10 repetitions per stimuli. FIG. 10B—Average firing rate for all the duration tested. FIG. 10C—Fraction of active sites at different stimulus duration for 4 different activity threshold. FIG. 10D—Time to first spike, average over 10 stimuli repetitions for all tested duration. Red dot mark the median value, edges of the box the 25th and 75th percentiles of the data, and whiskers the rest except for outliers plotted individually. The important drop of the median between 1 and 5 msec stimuli indicate that most recordings site start to respond for these duration. All stimuli were were presented at 600+/−20 nm, at an intensity of 2×10¹⁷ photons.cm⁻².s⁻¹.

FIG. 11: Effect of tdTomato on ChrimsonR mRNA levels. Amplification curves of ChrimsonR in a RT-qPCR reaction. The Y-axis represents the delta Rn value corresponding to an experimental reaction minus the Rn value of the baseline signal. This parameter reliably calculates the magnitude of the specific signal generated from a given set of PCR primers. Magenta and purple traces represent ChrimsonR; Yellow and orange traces represent ChrimsonR-tdTomato; Dark and light blue traces are the non-transfected controls. The experiment was repeated 3 times and each experiment was run on 2 plates yielding 6 total repetitions. Each sample was run in triplicates on each plate.

FIGS. 12A through 12B: Level of ChrimsonR protein upon transfection of HEK293 cells with pssAAV-CAG-ChrimsonR-tdTomato, pssAAV-CAG-ChrimsonR and pssAAV-CAG-ChrimsonR-GFP plasmids.

FIG. 13: Effect of tdTomato on the number of cells expressing ChrimsonR. Percentage of ChrimsonR-positive cells is represented for cells transfected with plasmid 479 (ChrimsonR-tdTomato) and 480 (ChrimsonR) compared to non-transfected controls. Percentage of fluorescent cells was determined by using a threshold value to eliminate background fluorescence. It is important to note that the number of cells does not indicate the intensity of fluorescence per cell. Based on this cell counting method there is no statistically significant difference between the percentage of ChrimsonR-expressing cells after transfection with the two constructs. Error bars represent SEM within this experiment and the experiment was repeated 3 times with technical duplicates for each condition.

FIGS. 14A through 14B: Effect of tdTomato on the subcellular localization of ChrimsonR in HEK 293T cells. Images of transfected HEK 293T cells; obtained by maximum projections of confocal z stacks. Cells nuclei are shown in blue (DAPI) and Chrimson R is shown in white. FIG. 14A shows localisation of Chrimson R-dtTomato; FIG. 14B shows distribution of ChrimsonR alone. Scale bars 20 μm.

FIGS. 15A through 15B: Effect of tdTomato on the subcellular localization of ChrimsonR in HEK 293T cells after AAV infection. Images of transfected HEK 293T cells; obtained by maximum projections of confocal z stacks. Cells nuclei are shown in blue (DAPI) and Chrimson R is shown in white. FIG. 15A shows localisation of Chrimson R-dtTomato; FIG. 15B shows distribution of ChrimsonR alone. Scale bars 20 μm.

### DETAILED DESCRIPTION

In this disclosure, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements and components comprising one unit and elements or components that comprise more than one unit unless specifically stated otherwise.

As used herein, the term “about,” when used in conjunction with a percentage or other numerical amount, means plus or minus 10% of that percentage or other numerical amount. For example, the term “about 80%,” would encompass 80% plus or minus 8%.

All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The terms “protein”, « polypeptide » and “peptide” as used herein are interchangeable, unless instructed to the contrary.

As used herein, the term « fusion protein » or « protein fused to another » refers to a protein construct or a chimeric protein. It is meant a single protein molecule containing two or more proteins or fragments thereof, covalently linked via peptide bond within their respective peptide chains, without additional chemical linkers. One protein can be fused to another protein either at the N-terminus or the C-terminus thereof. The fusion protein can further comprise linker moiety resulting from genetic construction.

As used herein, and unless otherwise indicated, the terms “treat,” “treating,” “treatment” and “therapy” contemplate an action that occurs while a patient is suffering from a disorder, e.g. a neuron mediated disorder or ocular disorders, that reduces the severity of one or more symptoms or effects of said disorder. As used herein, and unless otherwise indicated, the terms “prevent,” “preventing,” and “prevention” contemplate an action that occurs before a patient begins to suffer from a disorder, e.g. neuron mediated disorder or ocular disorder, that delays the onset of, and/or inhibits or reduces the severity of said disorder. It will be understood that a treatment may be a prophylactic treatment or may be a treatment administered following the diagnosis of a disease or condition. A treatment of the invention may reduce or eliminate a symptom or characteristic of a disorder, disease, or condition or may eliminate the disorder, disease, or condition itself. It will be understood that a treatment of the invention may reduce or eliminate progression of a disease, disorder or condition and may in some instances result in the regression of the disease, disorder, or condition. In some embodiments of the invention one or more light-activated ion channels polypeptide of the invention may be expressed in a cell population and used in methods to treat a disorder or condition.

As used herein, and unless otherwise specified, a “therapeutically effective amount” of a compound is an amount sufficient to provide any therapeutic benefit in the treatment or management of a neuron mediated disorder or ocular disorder, or to delay or minimize one or more symptoms associated with a disorder, e.g. a neuron mediated disorder or ocular disorders. A therapeutically effective amount of a compound means an amount of the compound, alone or in combination with one or more other therapies and/or therapeutic agents that provide any therapeutic benefit in the treatment or management of a disorder, e.g. a neuron mediated disorder or ocular disorders. The term “therapeutically effective amount” can encompass an amount that alleviates a neuron mediated disorder or ocular disorder, improves or reduces an ocular disorder, improves overall therapy, or enhances the therapeutic efficacy of another therapeutic agent.

As used herein, “patient” or “subject” includes mammalian organisms which are suffering or are susceptible to suffer from disorder as described herein, such as human and non-human mammals, for example, but not limited to, rodents, mice, rats, non-human primates, companion animals such as dogs and cats as well as livestock, e.g., sheep, cow, horse, etc.

Transfection of retinal neurons with nucleic acid (e.g. vector) encoding Chrimson polypeptide of the Invention provides retinal neurons, preferably bipolar cells and/or ganglion cells, with photosensitive membrane channels. Thus, it is possible to measure, with a light stimulus, the transmission of a visual stimulus to the animal's visual cortex, the area of the brain responsible for processing visual signals which constitutes a form of vision, as intended herein. Such vision may differ from forms of normal human vision and may be referred to as a sensation of light, also termed “light detection” or “light perception.” Thus, the term “vision” as used herein is defined as the ability of an organism to usefully detect light as a stimulus. “ Vision” is intended to encompass the following: (i) Light detection or perception, i.e. the ability to discern whether or not light is present (ii) Light projection, i.e. the ability to discern the direction from which a light stimulus is coming; (iii) Resolution, i.e. the ability to detect differing brightness levels (i.e., contrast) in a grating or letter target; and (iv) Recognition, i.e. the ability to recognize the shape of a visual target by reference to the differing contrast levels within the target. Thus, “vision” includes the ability to simply detect the presence of light, preferably red light, more preferably with light having a wavelength between about 365 nm and about 700 nm, between about 530 nm and about 640 nm, and in some embodiments, a peak activation may occur upon contact with light having a wavelength of about 590 nm.

As used herein, “Functional derivatives” encompass “mutants,” “variants” and “fragments” regardless of whether the terms are used in the conjunctive or the alternative herein. Preferred variants are single amino acid conservative substitution variants, though conservative substitution of 2, 3, 4 or 5 residues, for example, is also intended. In some embodiments, the Functional derivatives has at least 70% homology to the full length amino acid sequence of the original polypeptide, preferably at least 75%, more preferably at least 80% homology, more preferably at least 85% homology, more preferably at least 90% homology, more preferably at least 95% homology, more preferably at least 99% homology, more preferably 100% homology. The percent homology is determined with regard to the length of the relevant amino acid sequence. Therefore, if a polypeptide of the present invention is comprised within a larger polypeptide, the percent homology is determined with regard only to the portion of the polypeptide that corresponds to the polypeptide of the present invention and not the percent homology of the entirety of the larger polypeptide. “Percent homology” with reference to polypeptide sequences, refers to the percentage of identical amino acids between at least two polypeptide sequences aligned using the Basic Local Alignment Search Tool (BLAST) engine. See Tatusova et al. (1999) ibid. The BLAST engine is provided to the public by the National Center for Biotechnology Information (NCBI), Bethesda, Md. According to specific embodiments, the functional derivative is a polypeptide which comprises an amino acid sequence which has at least 70% homology to the full length sequence of the original polypeptide and wherein it only differs from its parent polypeptide by a substitution at one or more position(s). Said substitution is preferably «conservative substitution» or «semi conservative». In addition, or alternatively, the Functional derivatives has at least 70% identity to the full length amino acid sequence of the original polypeptide, preferably at least 75% identity, more preferably at least 80% identity, more preferably at least 85% identity, more preferably at least 90% identity, more preferably at least 95% identity, more preferably at least 99% identity, more preferably 100% identity. Methods of determining sequence identity or homology are known in the art.

As used herein, the term “conservative substitution” generally refers to amino acid replacements that preserve the structure and functional properties of a protein or polypeptide. Such functionally equivalent (conservative substitution) peptide amino acid sequences include, but are not limited to, additions or substitutions of amino acid residues within the amino acid sequences encoded by a nucleotide sequence that result in a silent change, thus producing a functionally equivalent gene product. Conservative amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

The invention in some aspects relates to the expression in cells of light-activated ion channel polypeptides that can be activated by contact with one or more pulses of light, which results in strong depolarization of the cell. Light-activated channel polypeptides of the invention, also referred to herein as light-activated ion channels can be expressed in specific cells, tissues, and/or organisms and used to control cells in vivo, ex vivo, and in vitro in response to pulses of light of a suitable wavelength.

As used herein, the term “ion channel” means a transmembrane polypeptide that forms a pore, which when activated opens, permitting ion conductance through the pore across the membrane.

According to the present invention, the light-activated ion channel polypeptide comprises Chrimson protein, or functional derivatives thereof, and a fluorescent protein.

According to the present invention, the light-activated ion channel polypeptide comprises Chrimson protein, or functional derivatives thereof, fused to a fluorescent protein.

According to special embodiment said Chrimson protein is selected in the group consisting in protein ChR88 (also referred to herein as Chrimson88—SEQ ID NO:1) or functional derivatives thereof, and K176R substituted Chrimson88 protein (also referred to herein as Chrimson88 protein with K176R substitution or ChrimsonR—SEQ ID NO: 2) or functional derivatives thereof.

According to the present invention, the light-activated ion channel polypeptide comprises (i) protein ChR88 (SEQ ID NO: 1) or functional derivatives thereof and (ii) a fluorescent protein.

According to preferred embodiment the light-activated ion channel polypeptide of the invention comprises (i) protein ChrimsonR (SEQ ID NO: 2) or functional derivatives thereof and (ii) a fluorescent protein.

According to special embodiment, the light-activated ion channel polypeptide of the invention consists of protein ChR88 (SEQ ID NO: 1) or functional derivatives thereof and fluorescent protein, both protein being expressed as independent proteins.

According to another embodiment the light-activated ion channel polypeptide of the invention consists of protein ChrimsonR (SEQ ID NO: 2) or functional derivatives thereof and fluorescent protein, both protein being expressed as independent proteins.

According to preferred embodiment, the light-activated ion channel polypeptide of the invention consists of protein ChR88 (SEQ ID NO: 1) or functional derivatives thereof fused to fluorescent protein.

According to more preferred embodiment the light-activated ion channel polypeptide of the invention consists of protein ChrimsonR (SEQ ID NO: 2) or functional derivatives thereof fused to fluorescent protein.

Light-activated ion channel polypeptides of the Invention are strongly activated by contact with red light, preferably with light having a wavelength between about 365 nm and about 700 nm, between about 530 nm and about 640 nm, and in some embodiments, a peak activation may occur upon contact with light having a wavelength of about 590 nm.

Contacting an excitable cell that includes a light-activated ion channel polypeptide of the invention with a light in the activating range of wavelengths strongly depolarizes the cell. Exemplary wavelengths of light that may be used to depolarize a cell expressing a light-activated ion channel polypeptide of the invention, include wavelengths from at least about 365 nm, 385 nm, 405 nm, 425 nm, 445 nm, 465 nm, 485 nm, 505 nm, 525 nm, 545 nm, 565 nm, 585 nm; 590 nm, 605 nm, 625 nm, 645 nm, 665 nm, 685 nm; and 700 nm, including all wavelengths therebetween. In some embodiments, light-activated ion channel polypeptides of the invention have a peak wavelength sensitivity in of 590 nm, and may elicit spikes up to 660 nm.

Light-activated ion channel polypeptides of the invention can be used to depolarize excitable cells in which one or more light-activated ion channels of the invention are expressed. In some embodiments, light-activated ion channel polypeptides of the invention can be expressed in a sub-population of cells in a cell population that also includes one or more additional subpopulations of cells that express light-activated ion channels that are activated by wavelengths of light that do not activate a light-activated ion channel polypeptide of the invention.

The peptide amino acid sequences that can be used in various embodiments include the light-activated ion channel polypeptide described herein (SEQ ID NOS: 1 or 2, or 5), as well as functionally equivalent polypeptides.

Such functionally equivalent peptide amino acid sequences (conservative substitutions) include, but are not limited to, additions or substitutions of amino acid residues within the amino acid sequences of the Invention, but that result in a silent change, thus producing a functionally equivalent polypeptide. Amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Conservative amino acid substitutions may alternatively be made on the basis of the hydropathic index of amino acids. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The use of the hydropathic amino acid index in conferring interactive biological function on a protein is understood in the art (Kyte and Doolittle, J. Mol. Biol. 157:105-132, 1982). It is known that in certain instances, certain amino acids may be substituted for other amino acids having a similar hydropathic index or score and still retain a similar biological activity. In making changes based upon the hydropathic index, in certain embodiments the substitution of amino acids whose hydropathic indices are within +−2 is included, while in other embodiments amino acid substitutions that are within +−1 are included, and in yet other embodiments amino acid substitutions within +−0.5 are included.

Conservative amino acid substitutions may alternatively be made on the basis of hydrophilicity, particularly where the biologically functional protein or peptide thereby created is intended for use in immunological embodiments. In certain embodiments, the greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0+−1); glutamate (+3.0+−1); serine (+0.3); asparagine (+0.2); glutamine (+0.2); glycine (0); threonine (−0.4); proline (−0.5+−1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments the substitution of amino acids whose hydrophilicity values are within +−2 is included, in certain embodiments those that are within +−1 are included, and in certain embodiments those within +−0.5 are included.

According to one preferred embodiment, the light-activated ion channel polypeptide of the invention is a fusion protein between a chrimson polypeptide (e.g. protein ChR88 or functional derivatives thereof, or protein ChrimsonR or functional derivatives thereof) and a fluorescent protein. The use of fusion proteins in which a polypeptide or peptide, or a truncated or mutant version of peptide is fused to an unrelated protein, polypeptide, or peptide, and can be designed on the basis of the desired peptide encoding nucleic acid and/or amino acid sequences described herein. In certain embodiments, a fusion protein may be readily purified by utilizing an antibody that selectively binds to the fusion protein being expressed.

In general, the retinal or retinal derivative necessary for the functioning of the light-activated ion channel polypeptide of the invention is produced by the cell to be transfected with said channel polypetide. However according to the invention, it is further disclosed a channelrhodopsin comprising a light-activated ion channel polypeptide of the invention and a retinal or retinal derivative such as for example 3,4-dehydroretinal, 13-ethylretinal, 9-dm-retinal, 3-hydroxyretinal, 4-hydroxyretinal, naphthyl retinal; 3,7,11-trimethyl-dodeca-2,4,6,8,10-pentaenal; 3,7-dimethyl-deca-2,4,6,8-tetraenal; 3,7-dimethyl-octa-2,4,6-trienal; as well as 6-7- or 8-9- or 10-11 rotation-blocked retinals (WO03084994).

While the desired peptide amino acid sequences described can be chemically synthesized (see, e.g., “Proteins: Structures and Molecular Principles” (Creighton, ed., W. H. Freeman & Company, New York, N.Y., 1984)), large polypeptides sequences may advantageously be produced by recombinant DNA technology using techniques well-known in the art for expressing nucleic acids containing a nucleic acid sequence that encodes the desired peptide. Such methods can be used to construct expression vectors containing peptide encoding nucleotide sequences and appropriate transcriptional and translational control signals. These methods include, for example, in vitro recombinant DNA techniques, synthetic techniques, and in vivo genetic recombination (see, e.g., “Molecular Cloning, A Laboratory Manual”, supra, and “Current Protocols in Molecular Biology”, supra). Alternatively, RNA and/or DNA encoding desired peptide encoding nucleotide sequences may be chemically synthesized using, for example, synthesizers (see, e.g., “Oligonucleotide Synthesis: A Practical Approach” (Gait, ed., IRL Press, Oxford, United Kingdom, 1984)).

The peptide amino acid sequences that can be used in various embodiments include the light-activated ion channel polypeptide described herein (SEQ ID NOS: 1 or 2, 5 or 6), as well as functionally equivalent peptides and functionally derivatives thereof, and their functional fragments. In fact, in some embodiments, any desired peptide amino acid sequences encoded by particular nucleotide sequences can be used, as is the use of any polynucleotide sequences encoding all, or any portion, of desired peptide amino acid sequences. The degenerate nature of the genetic code is well-known, and, accordingly, each light-activated channel polypeptide amino acid-encoding nucleotide sequence is generically representative of the well-known nucleic acid “triplet” codon, or in many cases codons, that can encode the amino acid. As such, as contemplated herein, the channelrhodopsin peptide amino acid sequences described herein, when taken together with the genetic code (see, e.g., “Molecular Cell Biology”, Table 4-1 at page 109 (Darnell et al., eds., W. H. Freeman & Company, New York, N.Y., 1986)), are generically representative of all the various permutations and combinations of nucleic acid sequences that can encode such amino acid sequences.

Some embodiments are isolated nucleic acid molecules comprising a nucleotide sequence that encodes a light-activated ion channel polypeptide of the invention. In some embodiments, the nucleotide sequence encodes polypeptide which comprises (i) protein ChR88 (SEQ ID NO: 1) or functional derivatives thereof, and (ii) a fluorescent protein. In another embodiments, the nucleotide sequence encodes polypeptide which comprises (i) protein ChrimsonR (SEQ ID NO: 2) or functional derivatives thereof, and (ii) a fluorescent protein.

According to one special embodiment, the nucleotide sequence encodes polypeptide which consists in protein ChR88 (SEQ ID NO: 1) or functional derivatives thereof, fused to fluorescent protein. According to preferred embodiment, the nucleotide sequence encodes polypeptide which comprises protein ChrimsonR (SEQ ID NO: 2) or functional derivatives thereof fused to fluorescent protein.

According to certain special embodiments, the fluorescent protein of the invention is selected from tdTomato (tdT) fluorescent protein and green fluorescent protein (GFP).

TdTomato is a bright red fluorescent protein (tdTomato's excitation peak 554 nm, peak of emission wavelength 581 nm) (Shaner NC et al., Nat Biotechnol, 22, 1567-1572, 2004). The genomic sequence encoding tdTomato according to the invention might show at least 84% identity with the synthetic construct tandem-dimer red fluorescent protein gene, complete cds (Genbank Accession number AY678269). According to a preferred embodiment, the encoded tdTomato protein moiety of the invention is a polypeptide having between about 70% and about 75%; or more preferably between about 75% and about 80%; or more preferably between about 80% and 90%; or even more preferably between about 90% and about 99% of amino acids that are identical to the amino acid sequence of SEQ ID NO:3.

In other embodiments, the present invention provides for an isolated nucleic acid encoding a polypeptide having between about 70% and about 75%; or more preferably between about 75% and about 80%; or more preferably between about 80% and 90%; or even more preferably between about 90% and about 99% of amino acids that are identical to the amino acid sequence of SEQ ID NO: 5 or fragments thereof.

Nucleic acid of the invention may include additional sequences including, but not limited to one or more signal sequences (e.g. enhancers, polyadenylation signals, additional restriction enzyme sites, multiple cloning sites) and/or promoter sequences, or other coding segments, or a combination thereof. The promoter can be inducible or constitutive, general or cell specific promoter. An example of cell-specific promoter is mGlu6-promoter specific of bipolar cells. Some embodiments are any of the disclosed methods wherein the promoter is a constitutive promoter. Some embodiments are any of the disclosed methods wherein the constitutive promoter includes, but is not limited to, a CMV promoter or CAG promoter (CAG promoter is hybrid cytomegalovirus (CMV) immediate early enhancer fused to the chicken beta-actin promoter (CBA) and SV40 intron insertion; Alexopoulou et al., BMC Cell Biol. 2008; 9: 2; SEQ ID NO: 8). Some embodiments are any of the disclosed methods wherein the promoter includes, but is not limited to, an inducible and/or a cell type-specific promoter. Selection of promoter, vectors, enhancers, polyadenylation sites is matter of routine design for those skilled in the art. Those elements are well described in literature and are commercially available.

In certain embodiments, the invention concerns isolated nucleic acid segments and recombinant vectors which encode a protein or peptide that includes within its amino acid sequence an amino acid sequence of light-activated ion channel polypeptide of the invention or a functional portions or variant thereof, such as those identified (e.g. SEQ ID NOS: 5).

In certain embodiments, the invention concerns isolated nucleic acid segments and recombinant vectors which comprises the amino acide sequence SEQ ID NO:6 or SEQ ID NO:7.

Some embodiments are recombinant nucleic acids comprising a nucleotide sequence that encodes amino acids of (i) SEQ ID NO: 1 or SEQ ID NO:2 with (ii) SEQ ID NO:3 or SEQ ID NO:4.

Some preferred embodiments are recombinant nucleic acids comprising a nucleotide sequence that encodes amino acids of SEQ ID NO:5 or fragments thereof.

Some preferred embodiments are recombinant nucleic acids comprising a nucleotide sequence SEQ ID NO:6 or SEQ ID NO:7.

Some embodiments are recombinant nucleic acids comprising a nucleotide sequence that encodes amino acids of (i) SEQ ID NO: 1 or SEQ ID NO:2, operably linked to a heterologous promoter and (ii) a nucleotide sequence that encodes amino acids of SEQ ID NO:3 or SEQ ID NO:4, operably linked to a heterologous promoter.

Some preferred embodiments are recombinant nucleic acids comprising a nucleotide sequence that encodes amino acids of SEQ ID NO:5 or fragments thereof, operably linked to a heterologous promoter.

Some preferred embodiments are recombinant nucleic acids comprising a nucleotide sequence SEQ ID NO:6 or SEQ ID NO:7, operably linked to a heterologous promoter.

Some preferred embodiments are recombinant nucleic acids comprising a nucleotide sequence SEQ ID NO:6 or SEQ ID NO:7, operably linked to CAG heterologous promoter (SEQ ID NO:8).

According to another aspect, the invention relates to a nucleic acid expression vector that includes a nucleic acid sequence that encodes any of the aforementioned light-activated ion channel polypeptides.

As used herein, the term “nucleic acid expression vector” refers to a nucleic acid molecule capable of transporting between different genetic environments another nucleic acid to which it has been operatively linked. The term “ vector ” also refers to a virus or organism that is capable of transporting the nucleic acid molecule. One type of vector is an episome, i.e., a nucleic acid molecule capable of extra-chromosomal replication. Some useful vectors are those capable of autonomous replication and/or expression of nucleic acids to which they are linked. Vectors capable of directing the expression of genes to which they are operatively linked are referred to herein as “expression vectors”. Expression vectors and methods of their use are well known in the art. Non-limiting examples of suitable expression vectors and methods for their use are provided herein. In preferred embodiment, the vector is suitable for gene therapy, more particularly for virus-mediated gene transfer. Examples of viruses suitable for gene therapy are retroviruses, adenoviruses, adeno-associated viruses (AAV), lentiviruses, poxviruses (e.g. MVA), alphaviruses, herpesviruses. However, gene therapy further encompasses non-viral methods such as use of naked DNA, liposomes associated with nucleic acids. Vectors useful in some methods of the invention can genetically insert light-activated ion channel polypeptides into dividing and non-dividing cells and can insert light-activated ion channel polypeptides to cells that are in vivo, in vitro, or ex vivo cells.

In some preferred embodiments, the nucleic acid expression vector comprising the gene for a light-activated ion channel of the invention is selected among AAV viral vectors. According to preferred embodiment said AAV viral vector is an AAV2 and more preferably is AAV2-7m8 viral vector (WO 2012/145601).

Some aspects of the invention include methods of treating a disorder or condition in a cell, tissue, or subject using light-activated ion channels polypeptide of the invention. Treatment methods of the invention may include administering to a subject in need of such treatment, a therapeutically effective amount of a light-activated ion channel polypeptide of the invention to treat the disorder.

Administration of a light-activated ion channel polypeptide of the invention may include administration pharmaceutical composition that includes effective amount of at least one light-activated ion channels polypeptide of the invention. Administration of a light-activated ion channel polypeptide of the invention may include administration pharmaceutical composition that includes a cell, wherein the cell expresses the light-activated ion channel of the invention. Administration of a light-activated ion channel polypeptide of the invention may include administration of effective amount of a pharmaceutical composition that includes a vector, wherein the vector comprises a nucleic acid sequence encoding the light-activated ion channel polypeptide of the invention and the administration of the vector results in expression of the light-activated ion channel polypeptide in a cell in the subject.

In some embodiments, are methods of treating or preventing a neuron mediated disorder, comprising: (a) delivering to a target cell a nucleic acid expression vector that encodes a light-activated ion channel polypeptides of the invention, expressible in said target cell, said vector comprising an open reading frame encoding the light-activated ion channel polypeptides of the invention, operatively linked to a promoter sequence, and optionally, a transcriptional regulatory sequence; and (b) expressing said vector in said target cell, wherein the expressed light-activated ion channel polypeptides activates said target cell upon exposure to light.

In some embodiments, the expressed light-activated ion channel polypeptide consists in protein ChR88 (SEQ ID NO: 1) or functional derivatives thereof, fused to fluorescent protein.

According to preferred embodiment, the expressed light-activated ion channel polypeptide consists in ChrimsonR (SEQ ID NO: 2) or functional derivatives thereof fused to fluorescent protein.

In preferred embodiments, the expressed light-activated ion channel polypeptide consists in protein ChR88 (SEQ ID NO: 1) or functional derivatives thereof, fused to fluorescent protein selected in the group consisting in tdTomato (tdT) fluorescent protein or green fluorescent protein (GFP).

According to preferred embodiment, the expressed light-activated ion channel polypeptide consists in ChrimsonR (SEQ ID NO: 2) or functional derivatives thereof fused to fluorescent protein selected in the group consisting in tdTomato (tdT) fluorescent protein (SEQ ID NO:3) or green fluorescent protein (GFP) (SEQ ID NO:4).

As used herein, and unless otherwise indicated, the term neuron mediated disorders for which the present methods and compositions may be used include, but are not limited to, neuronal dysfunctions, disorders of the brain, the central nervous system, the peripheral nervous system, neurological conditions, disorders of memory and learning disorders, cardiac arrhythmias, Parkinson's disease, ocular disorders, ear disorders, spinal cord injury, among others.

As used herein, and unless otherwise indicated, the term ocular disorders for which the present methods and compositions may be used to improve one or more parameters of vision include, but are not limited to, developmental abnormalities that affect both anterior and posterior segments of the eye. Anterior segment disorders include, but are not limited to, glaucoma, cataracts, corneal dystrophy, keratoconus. Posterior segment disorders include, but are not limited to, blinding disorders caused by photoreceptor degeneration, dysfunctioning, loss and/or death. Retinal disorders include retinitis pigmentosa (RP), macular deneneration (MD), congenital stationary night blindness, age-related macular degeneration and congenital cone dystrophies.

A target cell according to certain embodiments of the invention may be an excitable cell or a non-excitable cell. It is preferably a cell in which a light-activated ion channel polypeptide of the invention may be expressed and may be used in methods of the invention. It includes prokaryotic and eukaryotic cells. Target cells include but are not limited to mammalian cells. Examples of cells in which a light-activated ion channel polypeptide of the invention may be expressed are excitable cells, which include cells able to produce and respond to electrical signals.

Non-limiting examples of target cells according to the invention include neuronal cells (neurons), nervous system cells, cardiac cells, circulatory system cells, visual system cells, auditory system cells, secretory cells (such as pancreatic cells, adrenal medulla cells, pituitary cells, etc.), endocrine cells, or muscle cells. In some embodiments, a target cell used in conjunction with the invention may be a healthy normal cell, which is not known to have a disease, disorder or abnormal condition. In some embodiments, a target cell used in conjunction with methods and channels of the invention may be an abnormal cell, for example, a cell that has been diagnosed as having a disorder, disease, or condition, including, but not limited to a degenerative cell, a neurological disease-bearing cell, a cell model of a disease or condition, an injured cell, etc. In some embodiments of the invention, a cell may be a control cell.

According to one special embodiment, light-activated ion channel polypeptide of the invention may be expressed in cells from culture, cells in solution, cells obtained from subjects, and/or cells in a subject (in vivo cells). Light-activated ion channels may be expressed and activated in cultured cells, cultured tissues (e.g., brain slice preparations, etc.), and in living subjects, etc.

In a preferred embodiment, the target cell is mammalian cell and is an electrically excitable cell. Preferably, it is a photoreceptor cell, a retinal rod cell, a retinal cone cell, a retinal ganglion cell (RGC), an amacrine cell, a biporal neuron, a ganglion cell, a spiral ganglion neurons (SGNs), a cochlear nucleus neuron, a multipolar neuron, a granule cell, a neuron, or a hippocampal cell.

Some embodiments are methods of restoring light sensitivity to a retina, comprising: (a) delivering to a target retinal neuron a nucleic acid expression vector that encodes a light-activated ion channel polypeptides of the invention, expressible in said target retinal neuron, said vector comprising an open reading frame encoding the light-activated ion channel polypeptides of the invention, operatively linked to a promoter sequence, and optionally, a transcriptional regulatory sequence; and (b) expressing said vector in said target retinal neuron, wherein the expressed light-activated ion channel polypeptides renders said retinal neuron photosensitive, thereby restoring light sensitivity to said retina or a portion thereof.

One embodiment is a method of restoring light sensitivity to a retina wherein the expressed light-activated ion channel polypeptide consists in protein ChR88 (SEQ ID NO: 1) or functional derivatives thereof, fused to fluorescent protein.

One preferred embodiment is a method of restoring light sensitivity to a retina wherein the expressed light-activated ion channel polypeptide consists in ChrimsonR (SEQ ID NO: 2) or functional derivatives thereof fused to fluorescent protein.

One preferred embodiment is method of restoring light sensitivity to a retina wherein the expressed light-activated ion channel polypeptide consists in protein ChR88 (SEQ ID NO: 1) or functional derivatives thereof, fused to fluorescent protein selected in the group consisting in tdTomato (tdT) fluorescent protein or green fluorescent protein (GFP).

One preferred embodiment is method of restoring light sensitivity to a retina wherein the expressed light-activated ion channel polypeptide consists in ChrimsonR (SEQ ID NO: 2) or functional derivatives thereof fused to fluorescent protein selected in the group consisting in tdTomato (tdT) fluorescent protein (SEQ ID NO:3) or green fluorescent protein (GFP) (SEQ ID NO:4).

Some embodiments, are methods of restoring photosensitivity to a retina of a subject suffering from vision loss or blindness in whom retinal photoreceptor cells are degenerating or have degenerated and died, said method comprising: (a) delivering to a target retinal neuron a nucleic acid expression vector that encodes a light-activated ion channel polypeptides of the invention, expressible in said target retinal neuron, said vector comprising an open reading frame encoding the light-activated ion channel polypeptides of the invention, operatively linked to a promoter sequence, and optionally, a transcriptional regulatory sequence; and (b) expressing said vector in said target retinal neuron, wherein the expressed light-activated ion channel polypeptide renders said retinal neuron photosensitive, thereby restoring photosensitivity to said retina or a portion thereof.

Some embodiments, are methods of restoring photosensitivity to a retina of a subject suffering from vision loss or blindness in whom retinal photoreceptor cells are degenerating or have degenerated and died wherein the expressed light-activated ion channel polypeptide consists in protein ChR88 (SEQ ID NO: 1) or functional derivatives thereof, fused to fluorescent protein.

Some embodiments are methods of restoring photosensitivity to a retina of a subject suffering from vision loss or blindness in whom retinal photoreceptor cells are degenerating or have degenerated and died wherein the expressed light-activated ion channel polypeptide consists in ChrimsonR (SEQ ID NO: 2) or functional derivatives thereof fused to fluorescent protein.

Some preferred embodiments are methods of restoring photosensitivity to a retina of a subject suffering from vision loss or blindness in whom retinal photoreceptor cells are degenerating or have degenerated and died wherein the expressed light-activated ion channel polypeptide consists in protein ChR88 (SEQ ID NO: 1) or functional derivatives thereof, fused to fluorescent protein selected in the group consisting in tdTomato (tdT) fluorescent protein or green fluorescent protein (GFP).

Some preferred embodiments are methods of restoring photosensitivity to a retina of a subject suffering from vision loss or blindness in whom retinal photoreceptor cells are degenerating or have degenerated and died wherein the expressed light-activated ion channel polypeptide consists in ChrimsonR (SEQ ID NO: 2) or functional derivatives thereof fused to fluorescent protein selected in the group consisting in tdTomato (tdT) fluorescent protein (SEQ ID NO:3) or green fluorescent protein (GFP) (SEQ ID NO:4).

In some embodiments, the target neuron in said methods of treating a neuronal disorder, or of restoring light sensitivity to a retina, or of restoring photosensitivity to a retina of a subject suffering from vision loss or blindness in whom retinal photoreceptor cells are degenerating or have degenerated and died is a retinal neuron.

Some embodiments are any of the disclosed methods, wherein the expressed light-activated ion channel polypeptide having the amino acid sequence of all or part of SEQ ID NOS: 5, or a biologically active fragment thereof that retains the biological activity of the encoded light-activated channel polypeptide or a biologically active conservative amino acid substitution variant of SEQ ID NOS: 5 or of said fragment.

Some embodiments are any of the disclosed methods, wherein the expressed light-activated ion channel polypeptide is encoded by nucleic acid sequence SEQ ID NOS: 6.

Another aspect of the invention is the use of far-red (660 nm) light to perform non-invasive transcranial and/or transdural stimulation to modulate neural circuits.

Working operation of certain aspects of the invention was demonstrated by genetically expressing light-activated ion channel polypeptides of the invention in excitable cells, illuminating the cells with suitable wavelengths of light, and demonstrating rapid depolarization of the cells in response to the light, as well as rapid release from depolarization upon cessation of light. Depending on the particular implementation, methods of the invention allow light control of cellular functions in vivo, ex vivo, and in vitro.

In non-limiting examples of methods of the invention, light-activated ion channel polypeptides of the invention and derivatives thereof are used in mammalian cells without need for any kind of chemical supplement, and in normal cellular environmental conditions and ionic concentrations.

Light-activated ion channel polypeptides of the invention have been found to be suitable for expression and use in mammalian cells without need for any kind of chemical supplement, and in normal cellular environmental conditions and ionic concentrations. Light-activated ion channel polypeotides of the invention have been found to activate at wavelengths of light in a range of 365 nm to 700 nm, with an optimal activation from light ranging from 530 nm to 640 nm, and a peak optimal activation at a wavelength of 590 nm.

An effective amount of a light-activated ion channel polypeptide or of nucleic acid expression vector is an amount that increases the level of the light-activated ion channel in a cell, tissue or subject to a level that is beneficial for the subject. An effective amount may also be determined by assessing physiological effects of administration on a cell or subject, such as a decrease in symptoms following administration. Other assays will be known to one of ordinary skill in the art and can be employed for measuring the level of the response to a treatment. The amount of a treatment may be varied for example by increasing or decreasing the amount of the light-activated ion channel polypeptide or nucleic acid expression vector administered, by changing the therapeutic composition in which the light-activated ion channel polypeptide or nucleic acid expression vector is administered, by changing the route of administration, by changing the dosage timing, by changing the activation amounts and parameters of a light-activated ion channel of the invention, and so on. The effective amount will vary with the particular condition being treated, the age and physical condition of the subject being treated; the severity of the condition, the duration of the treatment, the nature of the concurrent therapy (if any), the specific route of administration, and the like factors within the knowledge and expertise of the health practitioner. For example, an effective amount may depend upon the location and number of cells in the subject in which the light-activated ion channel polypeptide is to be expressed. An effective amount may also depend on the location of the tissue to be treated. These factors are well known to those of ordinary skill in the art and can be addressed with no more than routine experimentation. It is generally preferred that a maximum dose of a composition to increase the level of a light-activated ion channel polypeptide, and/or to alter the length or timing of activation of a light-activated ion channel polypeptide in a subject (alone or in combination with other therapeutic agents) be used, that is, the highest safe dose or amount according to sound medical judgment. It will be understood by those of ordinary skill in the art, however, that a patient may insist upon a lower dose or tolerable dose for medical reasons, psychological reasons or for virtually any other reasons.

A light-activated ion channel polypeptide of the invention (for example, ChR88, or ChrimsonR fused with tdT or GFP, or a derivative thereof) may be administered using art-known methods. In some embodiments a nucleic acid that encodes a light-activated ion channel polypeptide of the invention is administered to a subject and in certain embodiments a light-activated ion channel polypeptide is administered to a subject. The manner and dosage administered may be adjusted by the individual physician or veterinarian, particularly in the event of any complication. The absolute amount administered will depend upon a variety of factors, including the material selected for administration, whether the administration is in single or multiple doses, and individual subject parameters including age, physical condition, size, weight, and the stage of the disease or condition. These factors are well known to those of ordinary skill in the art and can be addressed with no more than routine experimentation.

Pharmaceutical compositions that deliver light-activated ion channels polypeptide or nucleic acid expression vector of the invention may be administered alone, in combination with each other, and/or in combination with other drug therapies, or other treatment regimens that are administered to subjects. A pharmaceutical composition used in the foregoing methods preferably contain an effective amount of a therapeutic compound that will increase the level of a light-activated ion channel polypeptide to a level that produces the desired response in a unit of weight or volume suitable for administration to a subject.

The dose of a pharmaceutical composition that is administered to a subject to increase the level of light-activated ion channel polypeptide in cells of the subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The amount and timing of activation of a light-activated ion channel of the invention (e.g., light wavelength, length of light contact, etc.) that has been administered to a subject can also be adjusted based on efficacy of the treatment in a particular subject. Parameters for illumination and activation of light-activated ion channels that have been administered to a subject can be determined using art-known methods and without requiring undue experimentation.

Various modes of administration will be known to one of ordinary skill in the art that can be used to effectively deliver a pharmaceutical composition to increase the level of a light-activated ion channel polypeptide of the invention in a desired cell, tissue or body region of a subject. Methods for administering such a composition or other pharmaceutical compound of the invention may be topical, intravenous, oral, intracavity, intrathecal, intrasynovial, buccal, sublingual, intranasal, transdermal, intravitreal, subretinal, subcutaneous, intramuscular and intradermal administration. The invention is not limited by the particular modes of administration disclosed herein. Standard references in the art (e.g., Remington's Pharmaceutical Sciences, 18th edition, 1990) provide modes of administration and formulations for delivery of various pharmaceutical preparations and formulations in pharmaceutical carriers. Other protocols which are useful for the administration of a therapeutic compound of the invention will be known to one of ordinary skill in the art, in which the dose amount, schedule of administration, sites of administration, mode of administration (e.g., intra-organ) and the like vary from those presented herein.

Administration of a cell or vector to increase light-activated ion channel polypeptide levels in a mammal other than a human; and administration and use of light-activated ion channels of the invention. e.g. for testing purposes or veterinary therapeutic purposes, is carried out under substantially the same conditions as described above. It will be understood by one of ordinary skill in the art that this invention is applicable to both human and animals. Thus this invention is intended to be used in husbandry and veterinary medicine as well as in human therapeutics.

In some aspects of the invention, methods of treatment using a light-activated ion channel polypeptide of the invention are applied to cells including but not limited to a neuronal cell, a nervous system cell, a neuron, a cardiac cell, a circulatory system cell, a visual system cell, an auditory system cell, a muscle cell, or an endocrine cell, etc.

Disorders and conditions that may be treated using methods of the invention include, injury, brain damage, degenerative to neurological conditions (e.g., Parkinson's disease, Alzheimer's disease, seizure, vision loss, hearing loss, etc.

In some embodiments, methods and light-activated ion channels polypeptide of the invention may be used for the treatment of visual system disorders, for example to treat vision reduction or loss. A light-activated ion channel polypeptide of the invention or vector encoding such polypeptide may be administered to a subject who has a vision reduction or loss and the expressed light-activated ion channel can function as light-sensitive cells in the visual system, thereby permitting a gain of visual function in the subject.

Clinical applications of the disclosed methods and compositions include (but are not limited to) optogenetic approaches to therapy such as: restoration of vision by introduction of light-activated ion channels polypeptide of the invention in post-receptor neurons in the retina for ocular disorder gene-therapy treatment of age-dependent macular degeneration, diabetic retinopathy, and retinitis pigmentosa, as well as other conditions which result in loss of photoreceptor cells; control of cardiac function by using light-activated ion channels polypeptide of the invention incorporated into excitable cardiac muscle cells in the atrioventricular bundle (bundle of His) to control heart beat rhythm rather than an electrical pacemaker device; restoration of dopamine-related movement dysfunction in Parkinsonian patients; amelioration of depression; recovery of breathing after spinal cord injury; provide noninvasive control of stem cell differentiation and assess specific contributions of transplanted cells to tissue and network function.

Similarly, sensorineural hearing loss may be treated through optical stimulation of downstream targets in the auditory nerve (see Hernandez et al., 2014, J. Clin. Invest, 124 (3), 1114-1129 or Darrow et al., 2015,Brain Res., 1599, 44-56). According to special embodiment, the invention relates to methods of treating conductive hearing loss by the use of optical cochlear cochlear implants comprising: (a) delivering to cochlea a nucleic acid expression vector that encodes a light-activated ion channel polypeptides of the invention, expressible in said cochlea, said vector comprising an open reading frame encoding the light-activated ion channel polypeptides of the invention, operatively linked to a promoter sequence, and optionally, a transcriptional regulatory sequence; (b) expressing said vector in said cochlea, wherein the expressed light-activated ion channel polypeptides renders said cochlea photosensitive, and (c) use of a cochlear implant with flashes.

Some embodiments are methods of treating conductive hearing loss by the use of optical cochlear implants wherein the expressed light-activated ion channel polypeptide consists in protein ChR88 (SEQ ID NO: 1) or functional derivatives thereof, fused to fluorescent protein.

Some preferred embodiments are methods of treating conductive hearing loss by the use of optical cochlear implants wherein the expressed light-activated ion channel polypeptide consists in ChrimsonR (SEQ ID NO: 2) or functional derivatives thereof fused to fluorescent protein.

Some preferred embodiments are methods of treating conductive hearing loss by the use of optical cochlear implants wherein the expressed light-activated ion channel polypeptide consists in protein ChR88 (SEQ ID NO: 1) or functional derivatives thereof, fused to fluorescent protein selected in the group consisting in tdTomato (tdT) fluorescent protein or green fluorescent protein (GFP).

Some preferred embodiments are methods of treating conductive hearing loss by the use of optical cochlear implants wherein the expressed light-activated ion channel polypeptide consists in ChrimsonR (SEQ ID NO: 2) or functional derivatives thereof fused to fluorescent protein selected in the group consisting in tdTomato (tdT) fluorescent protein (SEQ ID NO:3) or green fluorescent protein (GFP) (SEQ ID NO:4).

The present invention in some aspects, includes preparing nucleic acid sequences and polynucleotide sequences; expressing in cells and membranes polypeptides encoded by the prepared nucleic acid and polynucleotide sequences; illuminating the cells and/or membranes with suitable light, and demonstrating rapid depolarization of the cells and/or a change in conductance across the membrane in response to light, as well as rapid release from depolarization upon cessation of light. The ability to controllably alter voltage across membranes and cell depolarization with light has been demonstrated. The present invention enables light-control of cellular functions in vivo, ex vivo, and in vitro, and the light activated ion channels of the invention and their use, have broad-ranging applications for drug screening, treatments, and research applications, some of which are describe herein.

In illustrative implementations of this invention, the ability to optically perturb, modify, or control cellular function offers many advantages over physical manipulation mechanisms. These advantages comprise speed, non-invasiveness, and the ability to easily span vast spatial scales from the nanoscale to macroscale.

The reagents use in the present invention (and the class of molecules that they represent), allow, at least: currents activated by light wavelengths not useful in previous light-activated ion channels, light activated ion channels that when activated, permit effectively zero calcium conductance, and different spectra from older molecules (opening up multi-color control of cells).

The following Examples section provides further details regarding examples of various embodiments. It should be appreciated by those of skill in the art that the techniques disclosed in the examples that follow represent techniques and/or compositions discovered by the inventors to function well. However, those of skill in the art should, in light of the present disclosure, appreciate that many changes can be made in the specific embodiments which are disclosed and still obtain a like or similar result without departing from the spirit and scope of the invention. These examples are illustrations of the methods and systems described herein and are not intended to limit the scope of the invention. Non-limiting examples of such include, but are not limited to those presented below.

### EXAMPLES

### Example 1

### Validation in rd1 and P23H Degenerative Rodent Models

Retinal dystrophies are associated with dysfunction and degeneration of retinal cells which impairs the flow of visual information, and ultimately leads to severe loss of vision and blindness. Retinitis pigmentosa (RP) is the most common type of retinal dystrophy and is responsible for loss of vision in one in 4,000 people worldwide. RP results from alteration in any of more than 60 genes inherited as autosomal dominant (30%-40% of cases), autosomal recessive (50%-60%), or X-linked (5%-15%).

In most common forms of RP, rod photoreceptors degenerate first followed by cones. Thus, primary symptoms of RP are usually night blindness and peripheral field loss leading to tunnel vision. All RP conditions are progressive and the pattern of sight deterioration varies amongst patients, however, the ultimate outcome is blindness. There is no treatment of RP.

Since RP results from multiple types of mutation in several genes, that a significant proportion of RP is dominant, and that time course of the disease is highly variable, a retinal optogenetic therapeutic approach is of potential interest. In this regard, retinal ganglion cells (RGCs) appear as an attractive target for the following reasons: 1) RGCs are firing cells whose axons directly project and carry visual information to visual cortical centers, 2) RGCs remained preserved in the macular region of RP patients even with advanced retinal degenerations 3) Retinal nerve fiber layer thickness is either reduced, increased or normal in RP patients 4) Clinical criteria for RGC optogenetic therapy can be readily assessed using OCT and scanning laser polarimetry. Photoreceptor degeneration leading to similar alteration of the retinal tissue is occurring in more complex retinal diseases such as age-related macular degeneration.

Optogenetic therapy of RGCs using channelrhodopsin-2 has proven to provide light-induced retinal electrical activity, visual evoked potentials and visual function, in rodent models of RP and normal monkey. In addition, since RGCs are closest to the vitreo-retinal surface, they are amenable to AAV infection with intravitreal injection, a major advantage from a surgical standpoint.

If ectopic expression of ChannelRhodopsin2 in retinal ganglion cells was shown to restore vision in blind rd1 mice, concerns on phototoxicity were raised by the required high excitation threshold in the blue wavelength range.

In this study, we investigated the use of ChrimsonR (ChrR), a red-shifted opsin, as radiation safety limits are much higher in the red light range. ChrimsonR is an enhanced form of the microbial opsin CnChR1 also named Chrimson or Chrimson 88, which was isolated from Chlamydomonas noctigama (Klapoetke et al., 2014, supra). Chrimson excitation spectrum is red-shifted by 45 nm relative to previous channelrhodopsins. ChrimsonR is K176R mutant of Chrimson, which exhibits a similar excitation spectrum but a better tetao_{ff} value (15.8 ms vs 21.4 ms). We have here investigated the use of ChrR for restoring vision in two degenerative models: blind rd1 mice and blind P23H rats.

During this study, we have compared further the functional efficacy of ChrR and the construct ChrimsonR-tdTomato (ChrR-tdT).

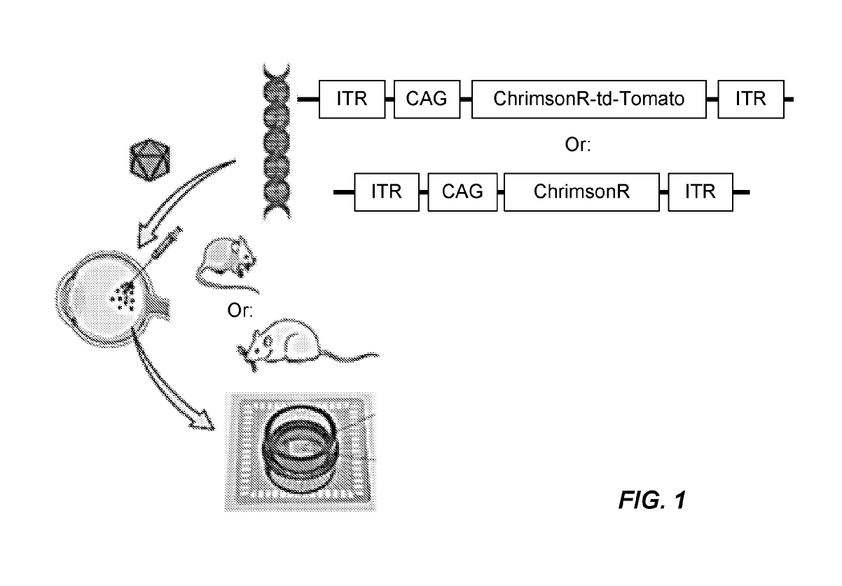

### Methods (FIG. 1)

### Gene Delivery

Virus batches used for mice experiments:

| | | | vol. | |
|---|---|---|---|---|
| prod | | titer | injected | |
| nb | production name | (vg/ml) | (μl) | vg/eye |
| 433 | AAV2.7m8-ssCAG-ChrimsonR | 2.25E+13 | 2 | 4.50E+10 |
| 432 | AAV2.7m8-ssCAG-ChrimsonR- | 1.54E+13 | 2 | 3.08E+10 |
| | tdTomato | | | |

Viral suspensions for GS030_NC_PHAR_007 Study were ready-to-use clear colourless liquids formulated in PBS+0.001% Pluronic® F68, in sterile 2-ml Eppendorf tube. Viral suspensions were made by dilutions from the stock viral suspensions with PBS+0.001% Pluronic® F68.

Viral suspensions were stored at 5±3° C. until use.

All experiments were done in accordance with the National Institutes of Health Guide for Care and Use of Laboratory Animals. The protocol was approved by the Local Animal Ethics Committees and conducted in accordance with Directive 2010/63/EU of the European Parliament.

4-week-old mice were anesthetized with isoflurane and intravitreal injection was performed bilaterally. In brief, pupils were dilated using tropicamide and the sclera was perforated using a needle near the limbus. A Hamilton syringe was then used to deliver 2 μl through a blunt injector into the eye.

Details of mouse injection and animal allocation:

| | Animal | | | Expression |
|---|---|---|---|---|
| | ID | Injection date | MEA date | time |
| ChrimsonR | 809OD | 23 Jan. 2015 | 19 Feb. 2015 | 27 |
| | 809OG | 23 Jan. 2015 | 19 Feb. 2015 | 27 |
| | 810OD | 23 Jan. 2015 | 10 Mar. 2015 | 46 |
| | 2304OD | 19 Feb. 2015 | 1 Apr. 2015 | 41 |
| | 2304OG | 19 Feb. 2015 | 1 Apr. 2015 | 41 |
| | 2303OG | 19 Feb. 2015 | 25 Mar. 2015 | 34 |
| ChrimsonR- | 875OD | 23 Jan. 2015 | 18 Feb. 2015 | 26 |
| tdTomato | 874OD | 23 Jan. 2015 | 20 Feb. 2015 | 28 |
| | 874OG | 23 Jan. 2015 | 20 Feb. 2015 | 28 |
| | 2301OG | 19 Feb. 2015 | 7 Apr. 2015 | 47 |
| | 2301OD | 19 Feb. 2015 | 7 Apr. 2015 | 47 |
| | 2302OG | 19 Feb. 2015 | 13 Apr. 2015 | 53 |
| | 2302OD | 19 Feb. 2015 | 13 Apr. 2015 | 53 |

### Retinal Preparation

Mice were sacrificed ˜5 weeks (27 to 53 days, average: 38 days) or 11 months after AAV injection by CO₂ inhalation followed by cervical dislocation. Animal eyeballs were isolated and dissected to remove the cornea and lens while keeping the retina attached to the sclera. This eye cup was conserved in a light tight container filled with Ames' solution (Sigma-Aldrich, St Louis, Mo.). Retina pieces (typically half a retina) were then isolated and use for multielectrode array recording.

### MEA Recordings

Multi-Electrode Array (MEA) recordings were obtained from ex-vivo mouse retina. The retinal fragments were placed on a cellulose membrane pre-incubated with polylysine (0.1%, Sigma) overnight. Once on a micromanipulator, the retinal piece was gently pressed against a MEA (MEA256 100/30 iR-ITO; Multi-Channel Systems, Reutlingen, Germany), with RGCs facing the electrode array. With the ChR-tdT construct, the fluorescence of tdTomato in the retinal piece on the electrode array was checked prior to recordings on the Nikon Eclipse Ti inverted microscope (Nikon, Dusseldorf, Germany) used to deliver the different light stimulations on the MEA system. The retina was continuously perfused with Ames' medium (Sigma-Aldrich, St Louis, Mo.) bubbled with 95% O₂ and 5% CO₂ at 34° C. at a rate of 1-2 ml/minute during experiments. A selective group III metabotropic glutamate receptor agonist, L-(+)-2-Amino-4-phosphonobutyric acid (L-AP4, 50 μM, Tocris Bioscience, Bristol, UK) was freshly diluted and bath applied through the perfusion system 10 minutes prior to recordings. Full field light stimuli were applied with a Polychrome V monochromator (Olympus, Hamburg, Germany) set to 600 nm (+/−15 nm), driven by a STG2008 stimulus generator (MCS). Output light intensities were calibrated to range from 1.37×10¹⁴ to 6.78×10¹⁶ photons.cm².sec⁻¹. For each light intensity, 10 repetitions of a 2-s flash is presented with 5-second interval between each stimulus. We also recorded responses to stimuli of variable durations using the polychrome (at the maximum light intensity, 6.78×10¹⁶ photons.cm².sec⁻¹) or using the source of a fluorescence microscope (X-cite, Lumen Dynamics) projecting on a digital micromirror display (DMD, Vialux, resolution 1024×768) coupled with a 600 +/−20 nm chromatic filter. Calibration indicated a light intensity of 2×10¹⁷ photons.cm².sec⁻¹ at the level of the retina. Single electrode activity was averaged over stimulus repetitions using an averaged spike density function (20 msec Gaussian standard deviation). Responsive electrodes are then averaged for each single retina.

### Immunohistochemistry and Imaging

Tissues were fixed for 30 min in 4% paraformaldehyde at room temperature. Saturation and permeabilization was done in a solution of PBS, bovine serum albumin (5%), Triton (0.5%) and Tween (0.25%) for one hour at room temperature. Incubation was done overnight at 4° C. in a diluted saturation solution (BSA 2.5%, Triton 0.25%, Tween 0.125%) with the primary antibody: 1/200 tdTomato. After four 20-min washes in PBS, tissues were incubated with secondary antibodies 1 h at room temperature. After five more PBS washes, tissues were mounted in vectashield and imaged using a confocal microscope (Olympus, Tokyo, Japan) equipped with 20× and 63× objectives.

### Results

### Localization of Transfected Cells

Five weeks after injection of ChrR-tdT, expression of the optogenetic protein,

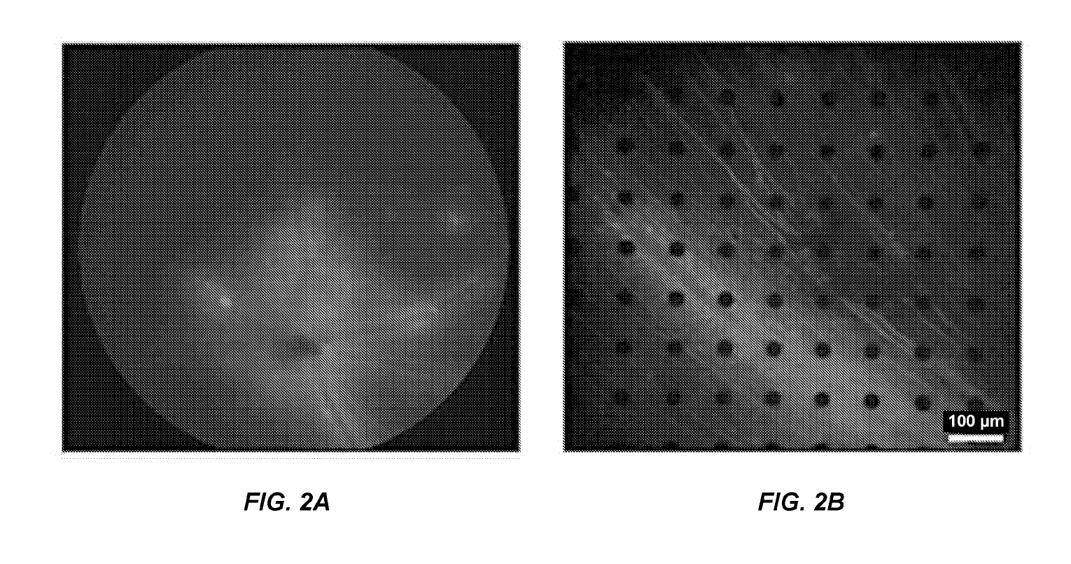

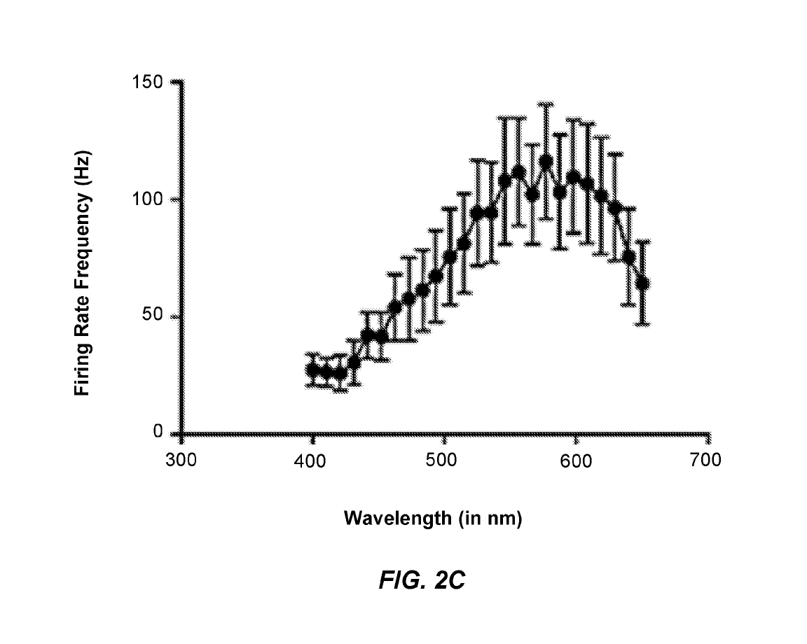

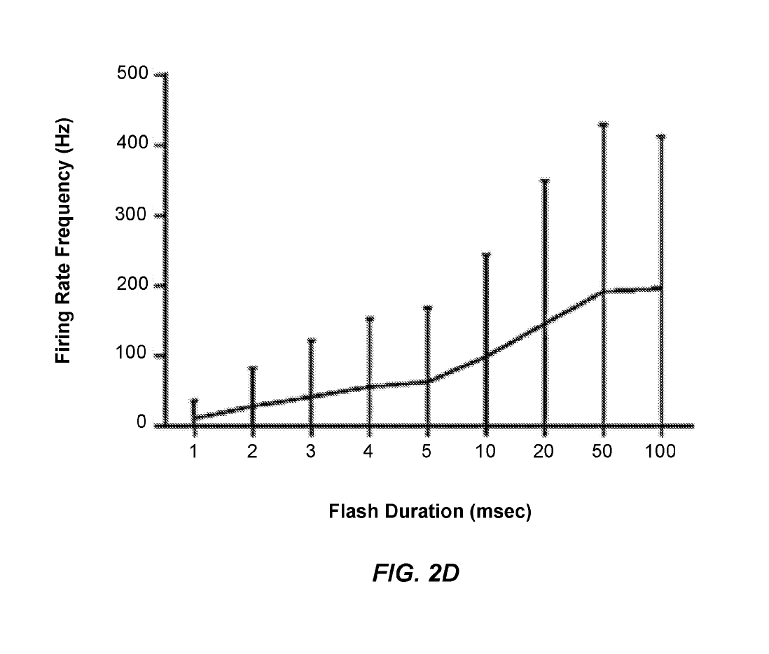

ChR, was readily visible thanks to tdTomato fluorescence. Its expression was found to be concentrated along large blood vessel present in the ganglion cells layer, as well as the optic disk (see FIG. 2A).

### MEA Recordings

To assess efficacy of ChrR and ChrR-tdT at a population level and without affecting cell integrity, we recorded transfected RGCs with a multielectrode array system (FIG. 2B). In order to avoid biasing the success rate of recordings toward the construct including the fluorescent reporter, tdTomato, the tissue fluorescence was examined after positioning the retinal piece on the electrode array (FIG. 2B). In addition, inhibition of potential light response originating from residual photoreceptors (Farber et al., 1994) was ensured through blockade of glutamate signaling (See Method Section).

For the two different conditions, animals were tested on one or two eyes.

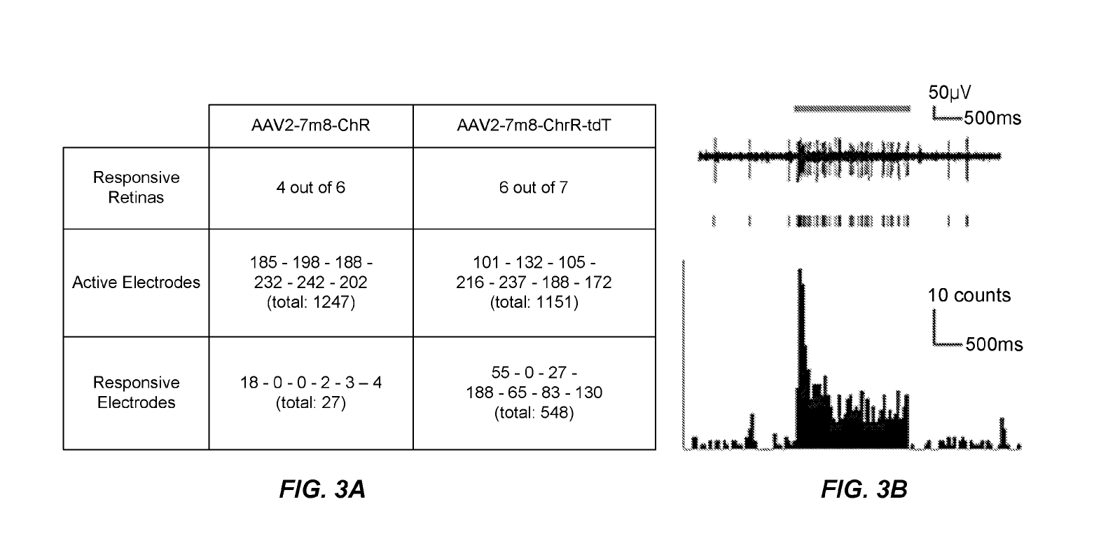

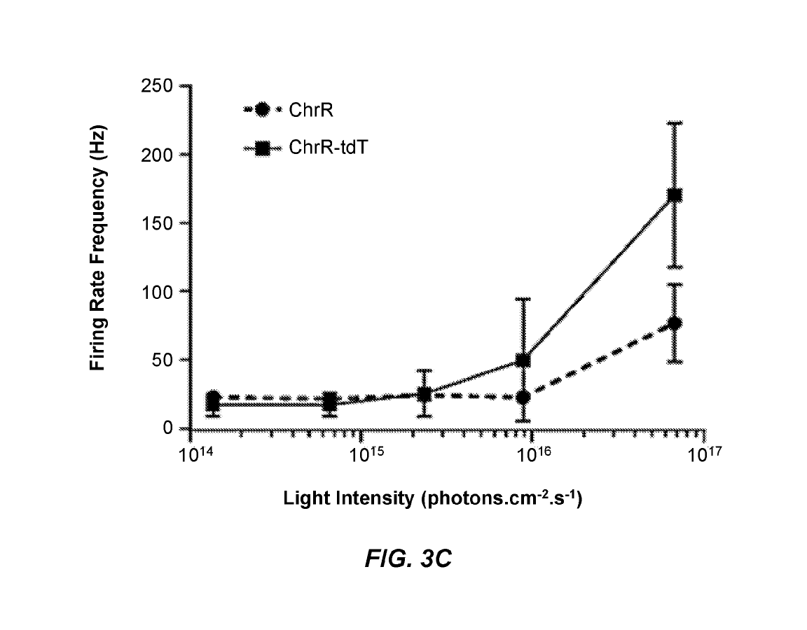

Recording was validated when a sufficient number of electrodes showed spontaneous RGC activity (FIG. 3A). This number of active electrodes range from 237 to 101. The ability to record spontaneous activity from a large number of electrodes is the hallmark of excellent experimental tissue conditions: 1) healthy retina and RGCs, and 2) adequate contact of the electrodes with the retinal tissue. Then, visual stimulation was generated at high light intensity in order to activate the microbial opsin, ChrR. In 6 of 7 eyes injected with ChrR-tdT and 4 of 6 eyes injected with ChrR construct, light-induced responses could be recorded (FIG. 3A-B). In responding retinas, the percentage of active electrodes recording an electrical activity upon light stimulation was determined. It reached 47% and 2% for ChrR-tdT and ChrR construct, respectively (FIG. 3A). These results suggest that ChrR-tdT is much more effective than ChrR construct to transform RGCs of rd1 mice into photosensitive cells.

### Sensitivity to Variable Light Intensity

600nm-light flashes were applied on the retinal tissue for 2 sec with a light intensity increasing from 1.37×1014 to 6.7833 1016 photons.cm2.sec-1. FIG. 2C the recorded responses with ChrR-tdT and ChrR constructs, respectively. Each line on the graph represents the plotted activity recorded at the responsive electrodes, where a light-elicited response was recorded at least for the highest light intensity.

These figures clearly illustrate that responses generated by the ChrR-tdT construct (FIG. 3C) were significantly greater in amplitude than ChrR at all intensities including the highest one. These recordings also show that the induced activity is mainly transient, with high peak values compared to the sustained amplitude. Finally, activation threshold seems to be lower with the ChrR-tdT construct, with first noticeable activity at 2.34×1015 photons.cm2.sec-1. Measuring the responses as the maximum added firing rate due to light stimulation, it confirms a lower threshold of response in ChrR-tdT expressing retina at 2.34×1015 photons.cm2.sec-1 and an activation at 8.82×1015 photons.cm2.sec-1 for ChrR construct (FIG. 3C). These observations indicate that the ChrR construct induced optogenetic responses with a higher intensity threshold and with lower spiking frequencies for a given intensity than those generated by the ChrR-tdT construct.

### Wavelength Sensitivity

In order to confirm the known light sensitivity of ChrimsonR, as well as to attest that the evoked activity is due only to ChrimsonR activity, we performed light stimulation over a full range of wavelengths (400 to 650 nm, FIG. 2C). As expected from published data (Klapoetke et al., 2014), peak firing was reached at 577-598 nm, consistent with a light sensitivity linked to ChrimsonR activation only.

### Expression Profile

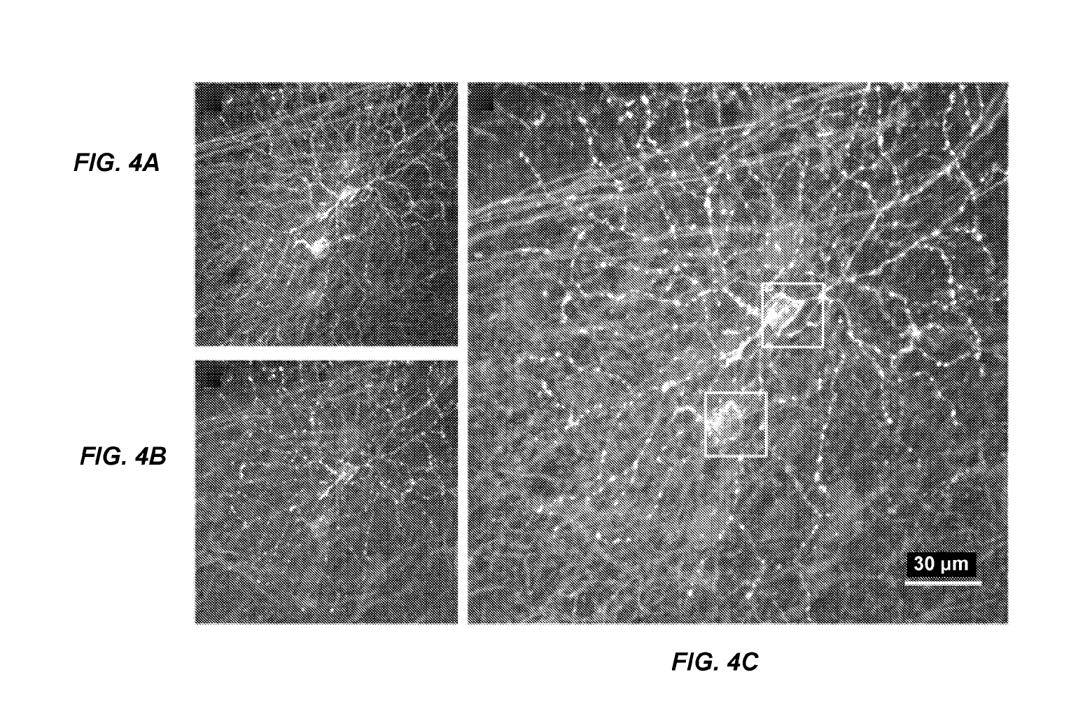

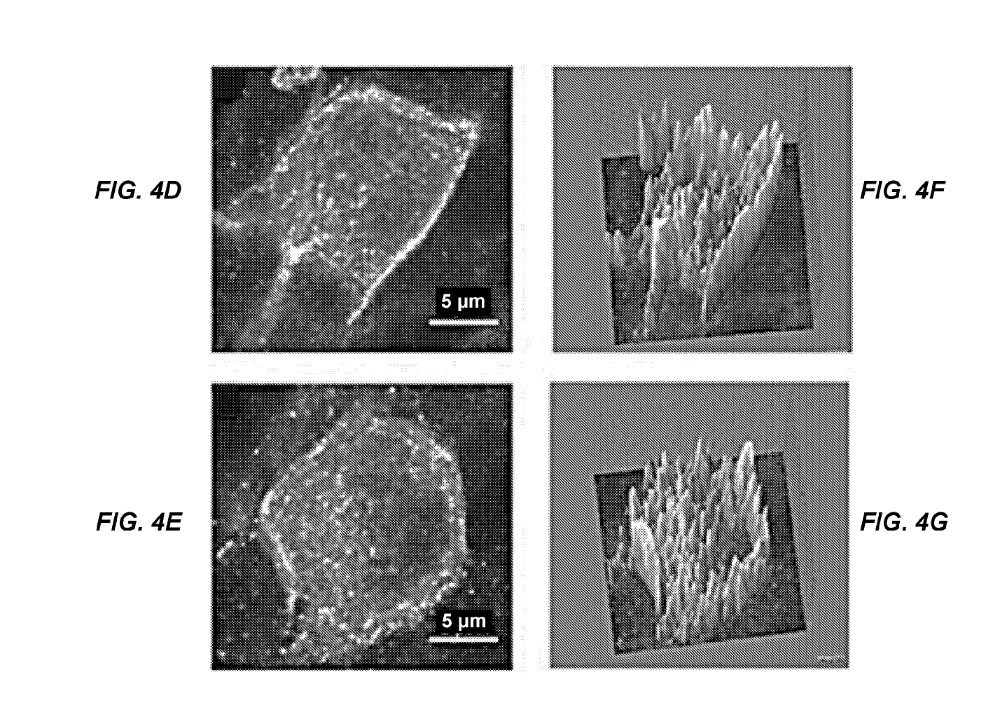

Expression in the retina was largely confined to cells of the ganglion cells layer, the innermost layer of the retina. Most of the cells expressing ChrR-tdT were retinal ganglion cells (RGCs) as indicated by their axons labelled by tdTomato (FIG. 4A-C). A close examination of cells expressing ChrR-tdT (FIG. 4D-E) revealed an enrichment of tdTomato fluorescence at, or near, the plasma membrane. Such a building-up of fluorescence at the cell membrane also occurred in cells with a relatively weak expression level. Finally, we had the opportunity to test a polyclonal antibody against ChrR (FIG. 4). ChrR antibody labelling confirmed that tdTomato-associated fluorescence is a good proxy for ChrimsonR localization.

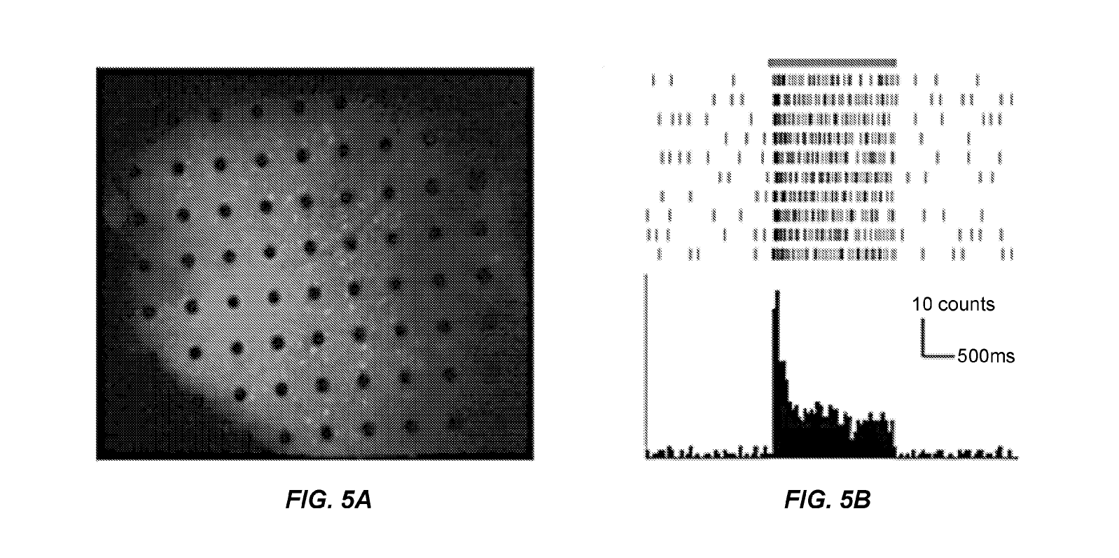

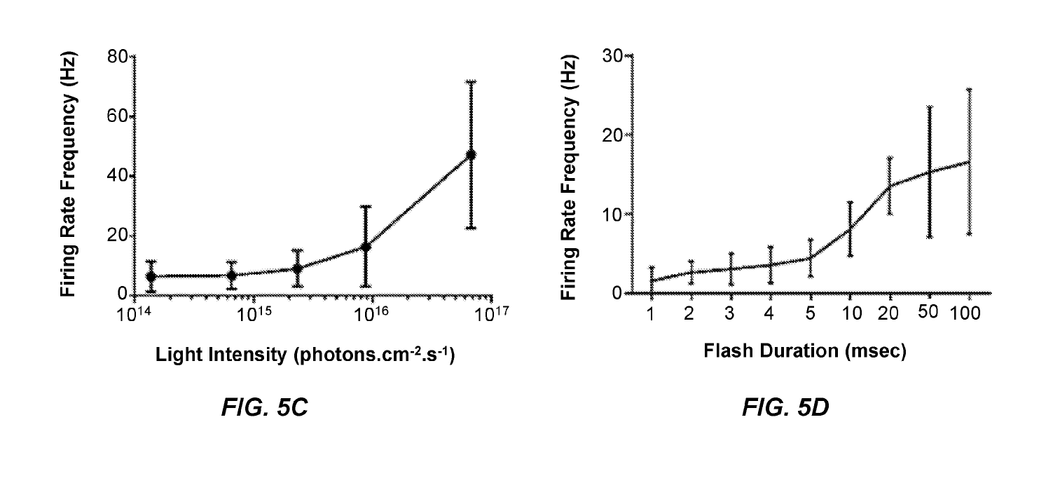

When rd1 mouse retina expressing ChrR-tdT were recorded 11 months after the viral vector injection (AAV2-7m8-ChrR-tdT), RGCs still produced major responses to light stimulation (FIG. 5) in areas with tdTomato expression (FIG. 5A). The sensitivities to light were similar to those recorded after 1 month of expression although lower amplitude responses were reached (FIG. 5C). These lower amplitude responses were attributed to the RGC degeneration occurring after the photoreceptor loss, which has been reported in animal models of retinitis pigmentosa and patients. Finally, the amplitudes of the responses were reaching a plateau at 20 ms in agreement with observations obtained at 1 month post injection (FIG. 5D). Therefore, these results indicated that the viral vector AAV2-7m8-ChrR-tdT can induce a long lasting expression of ChrR-tdT to drive the light response of RGCs in blind rd1 animals.

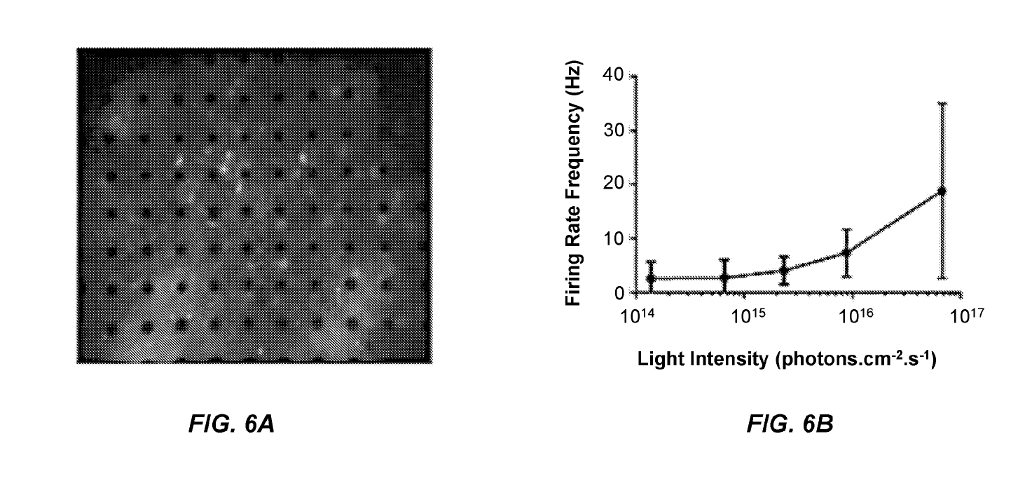

To further demonstrate the potential of ChrR-tdT expression in reactivating RGCs in different neurodegenerative models of photoreceptors, the viral vector (AAV2.7m8-ssCAG-ChrimsonR-tdTomato) was also injected intravitreally in P23H rats. MEA recording provided similar results in terms of RGC response amplitudes with respect to applied light intensities (FIG. 6). These results confirmed the interest for ChrR-TdT in photoreactivating RGCs following the loss of photoreceptors.

### Analysis

This study demonstrated the potential of ChrR for the reactivation of retinal ganglion cells in a blind retina of two different models of retinal degeneration. The data suggested that ChrR-TdT was much more potent than ChrR. ChrR-TdT could be activated at safe levels of light. These results paved the way for further preclinical investigation of ChrR-TdT expression and function in the non-human primate retina (see below).

### Example 2

### Activation of Retinal Ganglion Cell Populations in Non-Human Primates Below Safety Radiation Limits

In the study above, we had shown that ChrimsonR (ChrR), a red-shifted opsin, can induce light activation of retinal ganglion cells (RGCs) in blind rodents (rd1 mice and P23H rats). Furthermore, we had observed that the extended form ChrR fused to the fluorescent protein TdTomato appeared to provide a greater functional efficacy in terms of the number of cells responding to light and their response amplitudes. It is well established that, in contrast with rodents, AAV2 transduces only a ring of parafoveal RGCs in non-human primates (Yin et al., 2011). AAV2-7m8, extends beyond the foveal ring and leads to islands of expression in peripheral regions (Dalkara et al., 2013). A similar pattern of transduction with AAV2 vector is anticipated in humans.

Therefore, to further assess the translational potential of this therapeutic intervention, we assessed here in non-human primates whether an intravitreal injection of AAV vectors driving expression of ChrR, or ChrR fused to the fluorescent protein tdTomato (ChrR-tdT), can result in sufficient optogenetic protein expression to allow direct photoactivatation of RGCs.

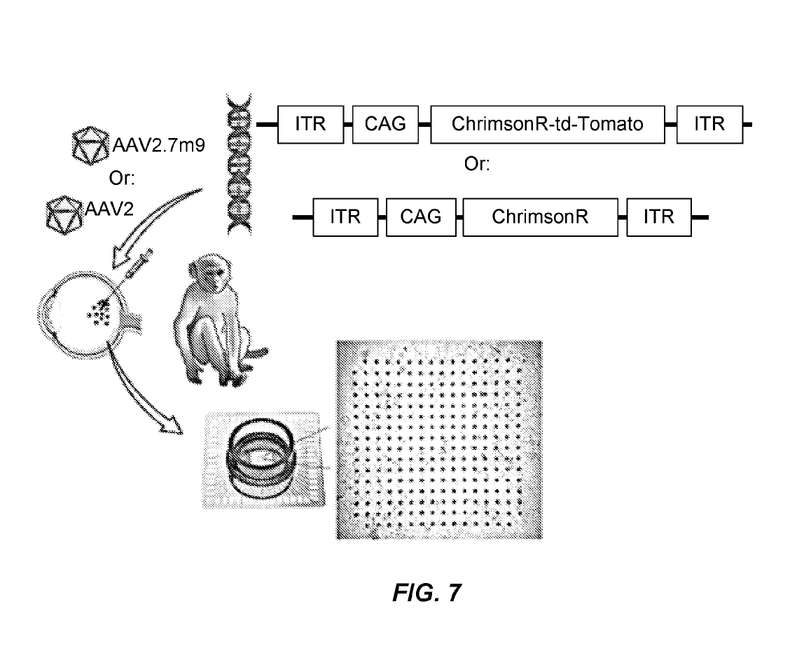

### Methods (See FIG. 7)

### Gene Delivery to the Primate Retina

### Virus Batches Used

| | | Diluted solution | | |
|---|---|---|---|---|
| # lot | Nom prod | vg/ml | vol | vg |
| 432 | AAV2.7m8-ssCAG- | 5 × 10e+12 | 400 | 2.00E+12 |
| | ChrimsonR-tdTomato | | | |
| 433 | AAV2.7m8-ssCAG- | 5 × 10e+12 | 400 | 2.00E+12 |
| | ChrimsonR | | | |
| 434 | AAV2-ssCAG-ChrimsonR- | 5 × 10e+12 | 400 | 2.00E+12 |
| | tdTomato | | | |
| 435 | AAV2-ssCAG-ChrimsonR | 5 × 10e+12 | 400 | 2.00E+12 |

The viral suspensions for GS030 study were ready-to-use clear colourless liquids formulated in PBS+0.001% Pluronic® F68, in sterile 2 ml Eppendorf tube. The viral suspensions were made by dilutions from the stock viral suspensions with PBS+0.001% Pluronic® F68.

The viral suspensions were stored at 5±3° C. until use.

| Viral | Injection | | |
|---|---|---|---|
| dose/eye | route | Right eye | Left eye |
| 5E11 vg | Intravitreal | AAV2-7m8- | AAV2-7m8-ChrimsonR- |
| | | ChrimsonR | tdTomato |
| 5E11 vg | Intravitreal | AAV2-7m8- | AAV2-7m8-ChrimsonR- |
| | | ChrimsonR | tdTomato |
| 5E11 vg | Intravitreal | AAV2-7m8- | AAV2-7m8-ChrimsonR- |
| | | ChrimsonR | tdTomato |
| 5E11 vg | Intravitreal | AAV2-7m8- | AAV2-7m8-ChrimsonR- |
| | | ChrimsonR | tdTomato |
| 5E11 vg | Intravitreal | AAV2-ChrimsonR | AAV2-ChrimsonR- |
| | | | tdTomato |
| 5E11 vg | Intravitreal | AAV2-ChrimsonR | AAV2-ChrimsonR- |
| | | | tdTomato |
| 5E11 vg | Intravitreal | AAV2-ChrimsonR | AAV2-ChrimsonR- |
| | | | tdTomato |
| 5E11 vg | Intravitreal | AAV2-ChrimsonR | AAV2-ChrimsonR- |
| | | | tdTomato |

### Primate Retina Isolation and Preservation

Two months (+/−5 days) after AAV injection, primates received a lethal dose of pentobarbital. Eyeballs were removed and placed in sealed bags for transport with CO2 independent medium (ThermoFisher scientific), after puncturation of the eye with a sterile 20-gauge needle. Retina were then isolated and conserved as retinal explants in an incubator for 12 to 36 hours prior to recording. Hemi-foveal retinal fragments were transferred on polycarbonate transwell (Corning) in Neurobasal+B27 medium for conservation in the cell culture incubator.

### MEA Recordings

Multitude Electrode Array (MEA) recordings were obtained from ex-vivo hemi-fovea retina. These retinal fragments were placed on a cellulose membrane pre-incubated with polylysine (0.1%, Sigma) overnight. Once on a micromanipulator, the retinal piece was gently pressed against a MEA (MEA256 100/30 iR-ITO; Multi-Channel Systems, Reutlingen, Germany), the retinal ganglion cells facing the electrodes. tdTomato fluorescence, when available, was checked prior to recordings with a Nikon Eclipse Ti inverted microscope (Nikon, Dusseldorf, Germany) mounted under the MEA system. The retina was continuously perfused with Ames medium (Sigma-Aldrich, St Louis, Mo.) bubbled with 95% O2 and 5% CO2 at 34° C. at a rate of 1-2 ml/minute during experiments. AMPA/kainate glutamate receptor antagonist, 6-cyano-7-nitroquinoxaline-2,3-dione (CNQX, 25 μM, Sigma-Aldrich), NMDA glutamate receptor antagonist, [3H]3-(2-carboxypiperazin-4-yl) propyl-1-phosphonic acid (CPP, 10 μM, Sigma-Aldrich) and a selective group III metabotropic glutamate receptor antagonist, L-(+)-2-Amino-4-phosphonobutyric acid (L-AP4, 50 μM, Tocris Bioscience, Bristol, UK) were freshly diluted and bath applied through the perfusion system 10 minutes prior to recordings. Full field light stimuli were applied with a Polychrome V monochromator (Olympus, Hamburg, Germany) driven by a STG2008 stimulus generator (MCS). Output light intensities were calibrated to range from 1.37×1014 to 6.78×1016 photons.cm2.sec-1. For each light intensity, the stimulus was applied for 2 seconds with 10-second interval between the 10 repetitions. The light spectrum sensitivity was generated by applying stimuli of 10 nm wavelength bandwidths from 400 to 650 nm in 10 nm steps for 2 seconds 10 times. The order of the tested wavelength bandwidths was randomized in order to prevent any adaptation of the retina. To define the minimum time required for eliciting a response, light stimuli were achieved with duration from 1 to 2,000 msec at the maximal light intensity, with 10 repetitions every 5 s.

### Results

### Localization of Transfected Cells

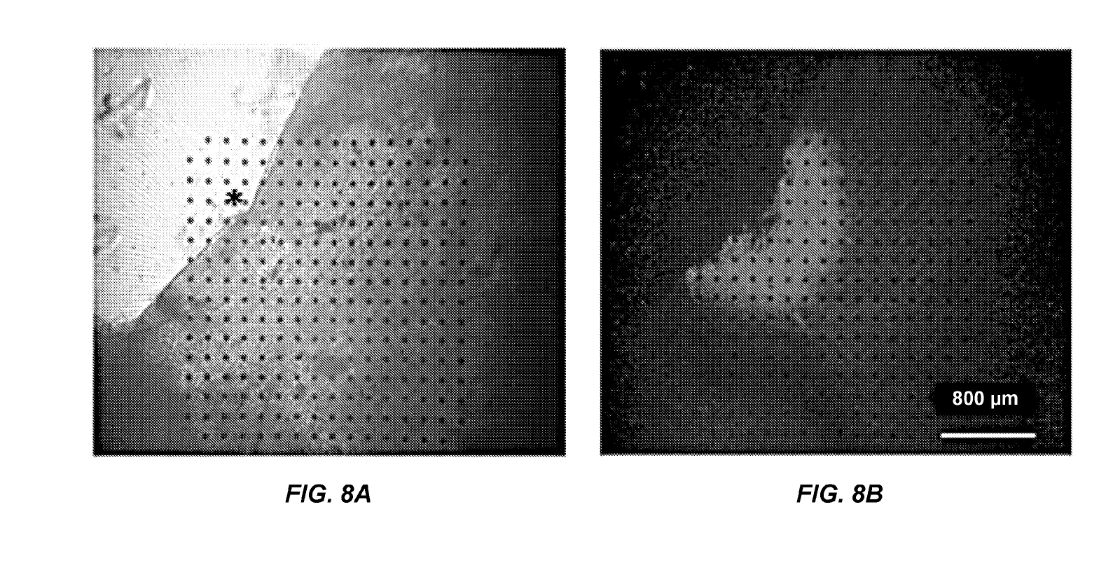

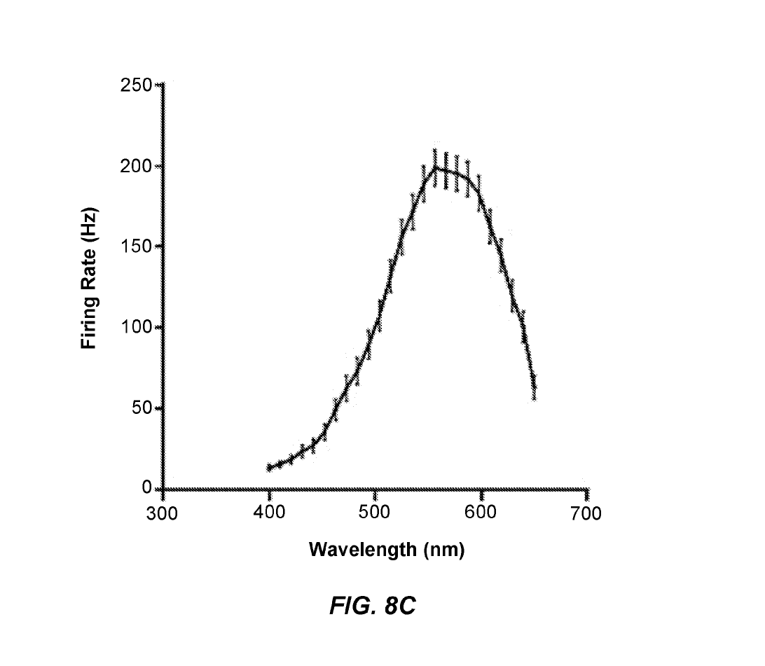

Previous studies on gene delivery following intravitreal injection of an AAV2 vector had shown the restriction of transfected cells to the foveal area especially the perifoveal ring of retinal ganglion cells (RGCs) (Dalkara et al., 2013). Therefore, when the retina was dissected out for recording RGCs, expression of tdTomato was examined in the retina with a greater attention toward this area. The fovea was cut in two halves for MEA recording. FIG. 8 illustrates the area with cells expressing tdTomato in the perifoveal ring on a flat-mounted retina, black dots represent electrodes of the MEA recording system. When the construct did not include tdTomato, the retina was similarly dissected and the foveal area similarly dissected out based on its identification using the yellow coloration of macular pigments.

### MEA Recordings

To assess efficacy of the different constructs at a large population level and without affecting cell integrity, we recorded transfected RGCs with a multielectrode array system (MEA). In all the 16 recorded NHP retina, we were able to record spontaneous activity from perifoveal RGCs (FIG. 8B). The number of “active” electrodes, where RGC spikes were spontaneously recorded, was consistently high (152 electrodes on average) with the exception of one AAV2.7m8-ChrimsonR experiment (only active 40 electrodes). The ability to record spontaneous activity from a large number of electrodes is the hallmark of excellent experimental conditions: 1) healthy retina and RGCs, and 2) adequate contact of the electrodes with the retinal tissue. When a light pulse was applied on the retina, an increase in spiking activity was measured on many electrodes (FIG. 8A). These electrodes were named responsive electrodes. Surprisingly, there were great differences among retina in terms of cells showing a light-evoked activity (FIG. 8B). Indeed, all retina (n=4) injected with AAV2.7m8-ChrR-TdT had responsive electrodes whereas all other groups had retina without responsive electrodes (AAV2.7m8-ChrR: ¼, AAV2-ChrR-TdT: 2/4, AAV2-ChrR: 0/4). It is worth mentioning that in case of an absence of the fluorescent marker TdTomato to localize the transfected cells, the retina was repositioned multiple times on the electrode array to increase the sampling area when no light response was measured.

### Light Sensitivity

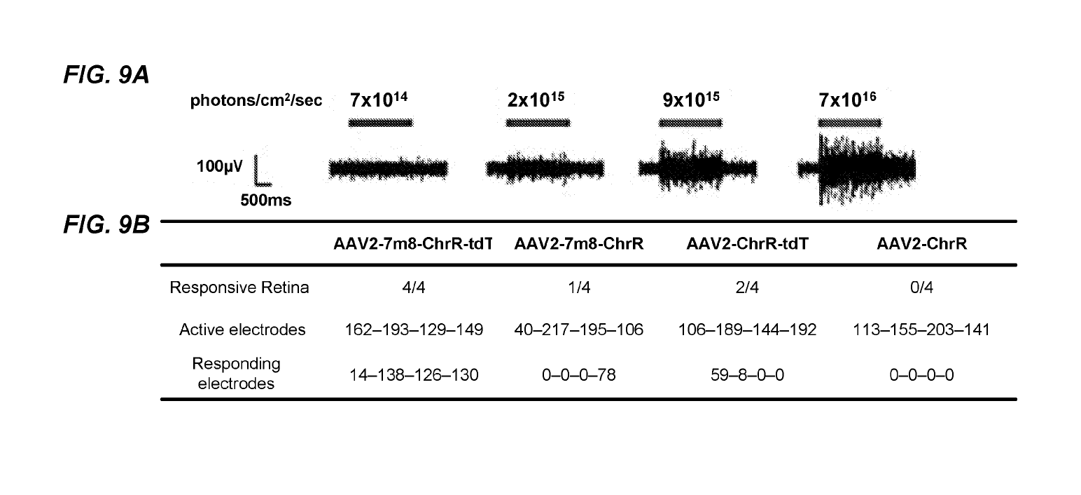

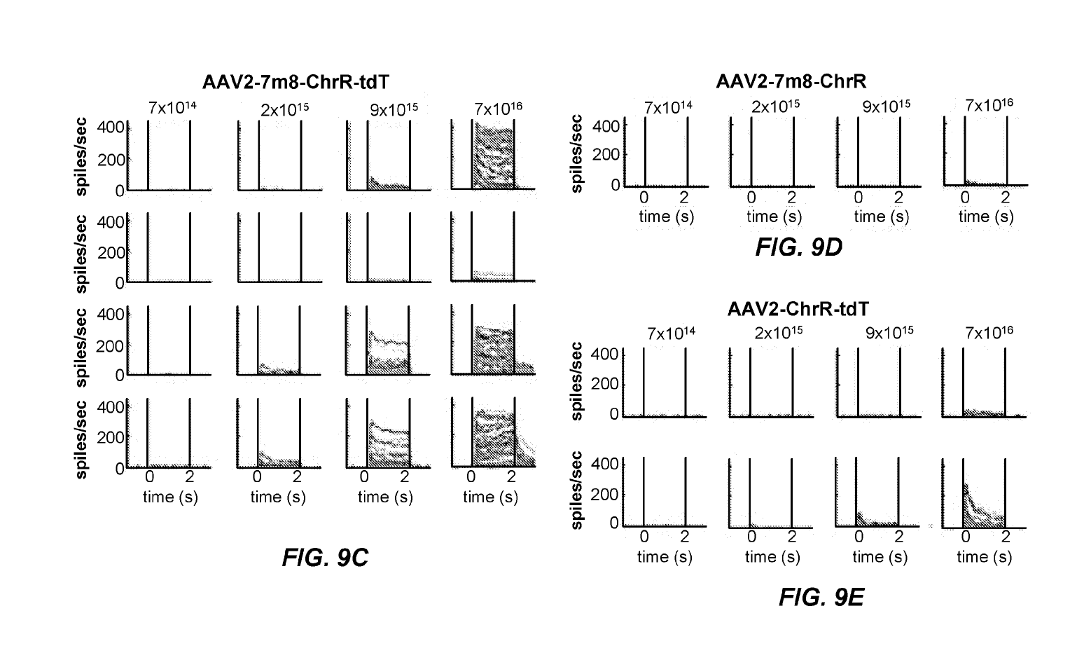

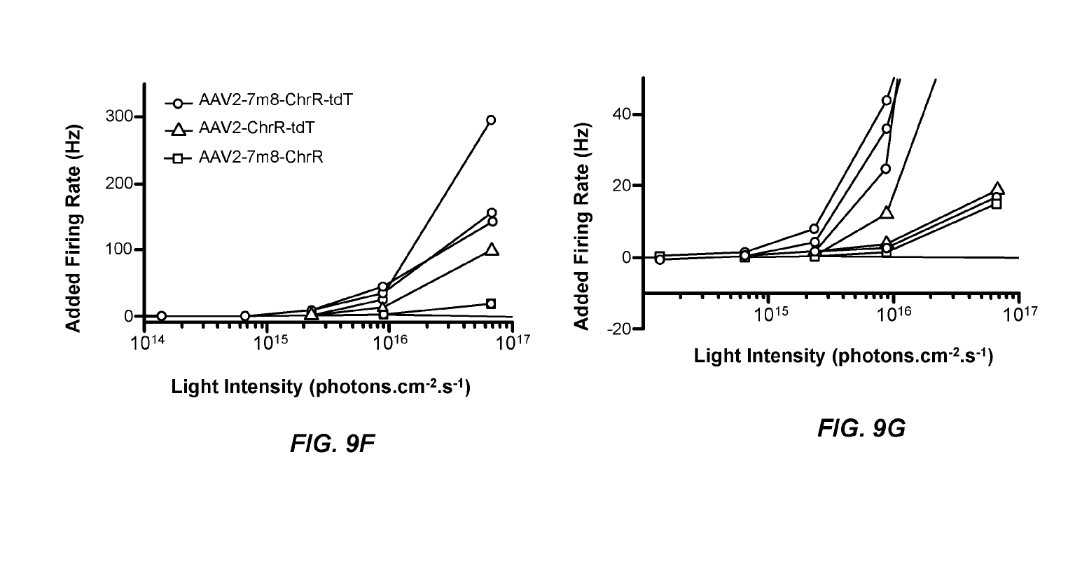

To detect a light response, light flashes were applied on the retinal tissue for 2 sec at 600 nm with a light intensity increasing from 1.37×1014 to 6.78×1016 photons.cm2.sec-1. FIG. 9A illustrates responses to different light intensities in a RGC from an eye injected with AAV2.7m8-ChrR-tdT. These light responses were then represented by spike rates with 50-msec bin widths (FIG. 9C). These responses not only displayed a strong sustained component but also often a transient component. FIGS. 9C-E represents the MEA recorded light responses for the different constructs under increasing light intensity. The amplitude of the responses increased with increasing light intensity although some variability was observed among the 4 different retinas with this best construct.

With the AAV2.7m8-ChrR-tdT construct, not only all retinas were light sensitive, but most retina showed higher response amplitudes (FIG. 9C). Furthermore, RGCs showed greater light sensitivities compared to other treatment groups (FIG. 9C-E). Two retinas displayed spike histograms of light responses at 2.34×1015 photons.cm2.sec-1 (FIG. 9C). At the highest light intensity tested, spiking frequencies at some electrodes were close to 400 Hz. FIGS. 9F-G provide graphs showing the amplitude of light response according to light intensities for various AAV constructs. Curves represent the average difference in cell firing rate during 2-sec stimuli minus the spontaneous firing rate. These two graphs are presented with two different Y axis scales in order to thoroughly show the full range of electrical response intensities while having a better illustration of response amplitude at low light levels. When ranking the different constructs according to their respective response amplitude, three retina transfected with AAV2.7m8-ChrR-tdT were much more sensitive than any other transfected retina. Of the two responsive AAV2-ChrR-tdT retinas, one came in the fourth position; the second responsive one being at a similar level than the sole responsive retina expressing AAV2.7m8-ChrR or the fourth retina expressing AAV2.7m8-ChrR-tdT. Therefore, AAV2.7m8-ChrR-tdT appeared as the most powerful construct with many more responsive retinas, greater sensitivities and with generally the highest amplitudes of electrical response.

### Action Spectrum

The light-induced electrical response at different wavelengths was measured for all retina displaying optogenetic light response. In this case, the action spectrum was established by quantifying the firing rate during the stimulus. When averaging the different action spectra measured for individual cells, we obtained an action spectrum of a single retina, which, by the way, was quite consistent with the ones obtained for mice above. FIG. 8C shows the spectrum of a retina injected with AAV2.7m8-ChrR-tdT. The peak of activity is reached at the peak of sensitivity of ChrimsonR (575 nm).

### Variable Duration Stimuli

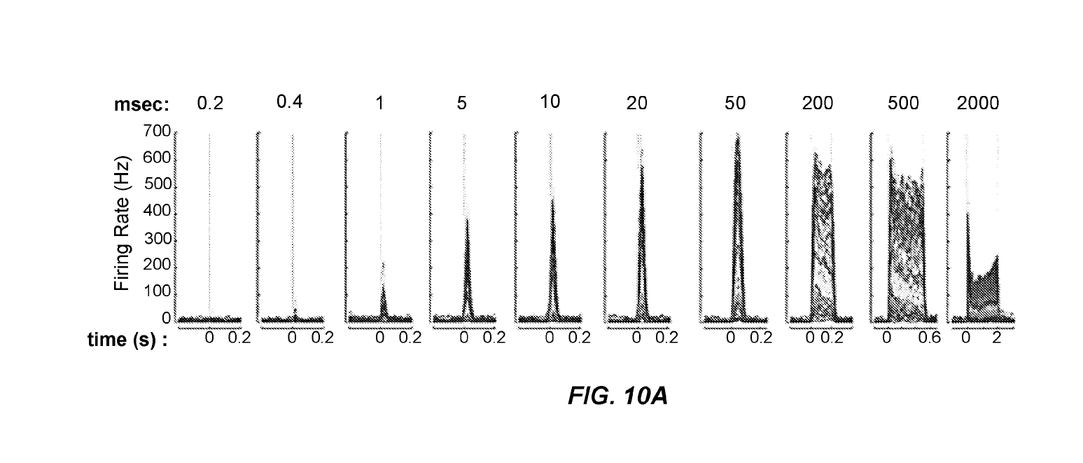

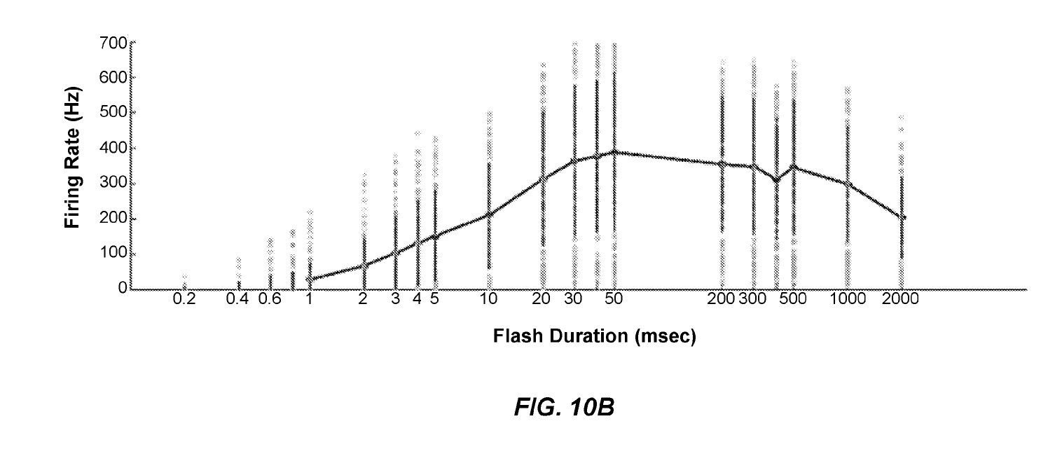

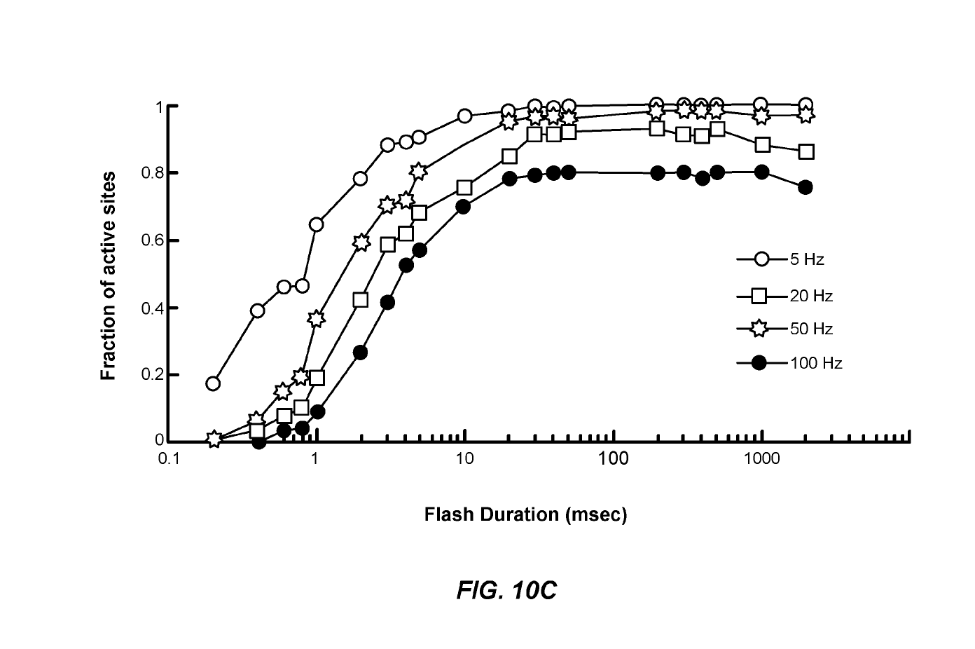

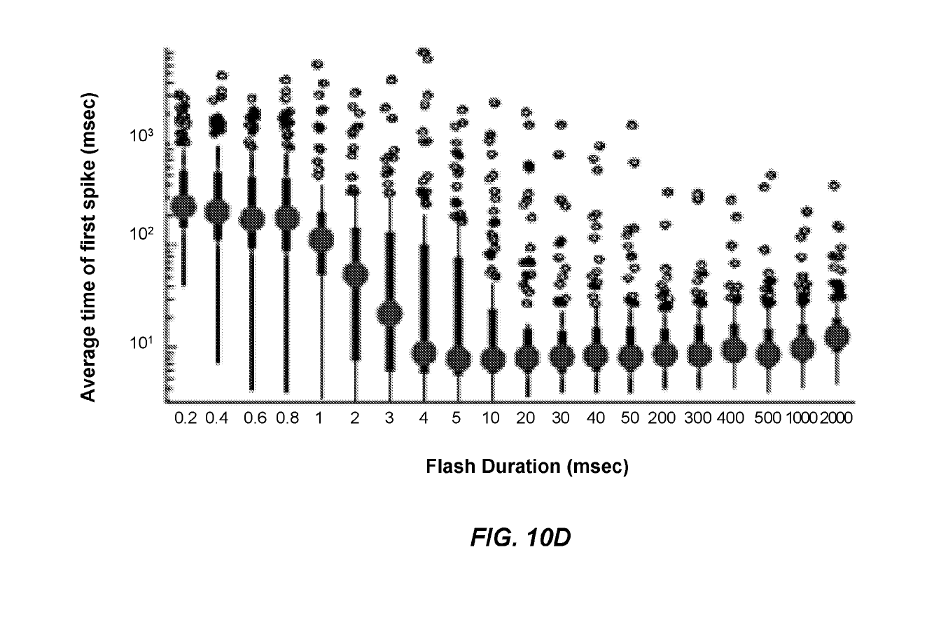

In order to determine the required stimulation duration to evoke a spiking behaviour, we applied stimuli of variable duration (from 0.2 msec to 2,000 msec) at high light intensity (using DMD as a source, 1.34×1018 photons.cm2.sec-1). FIG. 10 illustrates the data obtained for one retina injected with AAV2.7m8-ChrR-tdT. Light responses are displayed as a measured instantaneous firing rate for all responsive cells at all tested duration. The 2 second stimuli are used to define active electrodes based on an increased firing rate during the stimulations. Then, from all these active electrodes, responses to shorter stimuli were analyzed to examine the increase in spiking frequency during a window extending over the stimuli and 50 ms beyond. As can be seen on FIG. 10A-B, some cells displayed an increase in firing rate for stimuli as short as 0.4 msec. The number of responsive electrodes, as well as the instantaneous firing rate increased continuously for longer stimuli up to 50 ms. For longer stimuli, if the number of responsive cells does not change, the peak of instantaneous firing rates starts to decrease (FIG. 10A). To define the best stimulation parameters in a clinical setting, we assessed two important factors: the fraction of active sites for a given stimulation duration (FIG. 10C), and the average time to first spikes (FIG. 10D). The selected duration is expected to trigger activity in a sufficient number of potentially active cells with a fast dynamic (time to first spike). The fraction of active sites was defined for 4 different threshold values (5-20-50-100 Hz) of instantaneous firing rate. An electrode will be considered activated if the instantaneous firing rate during stimulation is higher than the considered threshold (the spontaneous firing rate was subtracted). FIG. 10C illustrates that, the added firing rate exceeded 5 Hz on more than 60% of the electrodes for 1 ms stimulus. In order to obtain a similar fraction of electrodes (roughly 70%) with an activity level above 100 Hz, stimuli of 10 ms are needed. We completed the analysis by measuring the average time to first spike for all sites and all durations. For this particular analysis, the spontaneous activity was not subtracted and it becomes very difficult to determine an accurate activation threshold for short duration eliciting no or a very low added spiking behavior. The long median values (˜200 msec) correspond in fact to the low spontaneous spiking rates of the cells (˜5 Hz) (0.2-1 ms, FIG. 10D). For longer stimuli duration (4-10 msec), the median values for the average time to first spike reached a plateau. These data indicate that, at this particular light intensity, 10 ms will provide fast response kinetics at a high rate of activity in more than half of the responsive cells. Therefore, these characteristics are compatible with at least a video rate activation of the retinal ganglion cells indicating thereby that AAV2.7m8-ChrR-tdT would provide an expression adequate for visual perception.

### Analysis

The capacity of three constructs (AAV2.7m8-ChrR-tdT, AAV2.7m8-ChrR, and AAV2-ChrR-tdT) to turn light-insensitive RGCs into photoactivable cells following intravitreal injection was investigated in the macaque monkey.

First, our data reproduced previous findings showing a specific infection of RGCs within the perifoveal ring following intravitreal administration of AAV2. However, and in line with Dalkara et al. (2013), infection rate was apparently stronger with AAV2.7m8 than with conventional AAV2. MEA were used to characterize functional response of RGCs to 600 nm light in flat-mounted retina two months after intravitreal injection. Results clearly established that AAV2-7m8-ChrR-tdT is the best candidate out of the four tested constructs, both regarding level of expression and functional activity. In this regard, 3 out of 4 retinas expressing ChrR-tdT produced large photocurrents and high frequencies of firing in response to illumination. Only one out of four retinas treated with AAV2.7m8-ChrR responded to light indicating that fusion of ChrR with tdTomato markedly enhances the function of the optogenetic protein.

In this study, we have established the light intensity range requested to evoke stimulation of ChrR-tdT-engineered RGCs. Analysis of photocurrents evoked by ChrR in RGCs at different light intensity provides valuable information on the kinetics of ChrR activation and inactivation. A 10 msec stimulation was shown to recruit a large number of responsive cells generating a high spiking rate with a fast kinetics. Action spectrum of the optogenetic protein was established and showed that maximal response of ChrimsonR-tdTomato construct was at about 575 nm wavelength. Taken together, these results allow selecting AAV2.7m8-ChrR-tdT as a candidate for restoring vision in patients.

### Example 3

### Role of the Fluorescent Protein tdTomato in Expression and Localization of the Optogenetic Protein ChrimsonR

In non-human primates and retinitis pigmentosa-bearing rd1 mice, AAV2.7m8-CAG-ChrimsonR-tdTomato was substantially more potent than a similar construct lacking tdTomato (AAV2.7m8-CAG-ChrimsonR). Thus, we aimed at understanding the underlying mechanism. To do so, in vitro studies in HEK293 cells were conducted focusing on expression and trafficking of ChrimsonR alone or fused with tdTomato.

### Methods

Human HEK293 cells were seeded in 24-well plates in a DMEM medium supplemented with 10% fetal calf serum. Cells were used at 10 to 70% confluence and between passage 3 and 20. Cell transfection of pssAAV-CAG-ChrimsonR-tdTomato, pssAAV-CAG-ChrimsonR and pssAAV-CAG-ChrimsonR-GFP plasmids was achieved using jetPrime® as a transfection agent (1 μl of jetPrime® mixed to 0.5 μg of plasmid DNA in 50 μl buffer solution).

ChrimsonR, ChrimsonR-tdTomato and ChrimsonR-GFP mRNA expression was examined by RT-PCR, and actin house-keeping gene mRNA expression ran in parallel. Cell level of fluorescence corresponding to ChrimsonR protein amount was evaluated by immunochemistry. An anti-ChrimsonR antibody belonging to and provided by GenSight was used at 1:1,000 dilution. A secondary anti-mouse antibody coupled to Alexafluor was used for immunofluorescence quantitation.

### HEK 293T Cell Culture

HEK 293T (ATCC® CRL-3216™) cells were maintained between 10% and 70% confluence in DMEM medium (Invitrogen, Waltham, USA) supplemented with 10% FBS (Invitrogen), 1% penicillin/streptomycin (Invitrogen).

### Transfections and Infections

Transfection of cells with pssAAV-CAG-ChrimsonR-tdTomato (plasmid 479), and pssAAV-CAG-ChrimsonR (plasmid 480) was done using jetPrime® as a transfection reagent (http://www.polyplus-transfection.com/products/jetprime/). A 24-well plate was prepared with a glass coverslip at the bottom of each well. Glass coverslips were coated with Poly-D-Lysine and Laminin HEK 293T cells were plated one day prior to transfection in these 24-well plates, at a density of 100,000 cells per well. One μl of jetPrime was mixed with 0.5 μg of plasmid DNA 479 or 480 in 50 μl buffer solution. 51.5 μl transfection mix was added to the cells and media was changed 4-6 hours after transfection. Cells were then incubated 24 hours after transfection prior to analysis.

For infections, cells were prepared as described above (plated one day prior to transfection in 24-well plates, at a density of about 100,000 cells per well). The next day, cells in one well were trypsinized and counted to determine the exact number of cells/well to calculate MOI. Cells are then infected with at a MOI of 500,000 with AAV2-7m8-CAG-ChrimsonR-tdTomato (IDV_lot 768) or with AAV2-7m8-CAG-ChrimsonR (IDV lot 752). 24-hours post-infection cells were fixed with 4% PFA.

### RT-qPCR

RNA was extracted from cellular lysates with the Nucleospin® RNA kit (Macherey-Nagel). Briefly, cells were lysed using the provided reagents, and lysate was filtered to remove cell debris. RNA was linked to a silica membrane. Contaminating DNA was degraded by nebulization and by the action of a DNAse. RNA was washed and eluted in RNAse free water. The RNA concentration and purity was assayed by UV spectrometry using Nanodrop. One μg was deposited on a 1% agarose gel in the presence of 1 kb size marker to assess RNA quality. RNA was then treated with a second DNAse: TURBO® DNAse (2 U of TURBO DNAse per reaction is added and followed by a 20-30 min incubation at room temperature (RT)) and 1 ng of RNA was used for RT-qPCR. Reverse transcription was done using the universal oligo dT primers. Specific qPCR was done with primers matching parts of ChrimsonR sequence (Primer Actin Forward: GCTCTTTTCCAGCCTTCCTT (SEQ ID NO:9), Primer Actin Rev: CTTCTGCATCCTGTCAGCAA(SEQ ID NO:10), Primer ChrimsonR, Forward: ACACCTACAGGCGAGTGCTT(SEQ ID NO:11), Primer ChrimsonR Rev: TCCGTAAGAAGGGTCACACC (SEQ ID NO:12). Standardization was done against the actin encoding housekeeping gene. Relative analysis method was used (a standard range with an equimolar mixture of the reverse transcript samples was prepared and diluted sequentially in 1:10 increments). Each dilution of the standard was dispatched in triplicates on the qPCR plate before mixing with the above-mentioned primers. Relative expression analysis was conducted subsequently. The RT-qPCR was repeated two times (on two 96-well plates) and each transfection condition was tested in triplicates.

### Immunohistochemistry

Cells were rinsed with PBS and fixed with 4% PFA for 10 minutes at room temperature. Blocking buffer (PBS with 1% Triton X-100, 0.5% Tween 20 and 10% BSA blocking buffer) was added for 15 minutes at Room Temperature. Cells were then incubated at RT for 2 hours with mouse polyclonal antibody directed against ChrimsonR (0.59 mg/mL) diluted at 1:1,000 in blocking buffer (10% BSA, 1% Triton X-100, 0.5% Tween). Three PBS washes were performed. Cells were then incubated with secondary anti-mouse antibodies coupled to AlexaFluor 488 (A-31571 Thermo fisher produced in donkey, dilution 1:500) for 1 hour at RT. The experiment was done 3 times in 3 replicates.

### Array Scan Imaging and Quantification

HEK 293T cells were transfected or infected as described above. Antibodies against ChrimsonR were applied to treated and control wells as described above. Cell nuclei were stained with Hoechst nuclear dye for 5 min then washed and imaged on the Cellomics Array Scan VTI. Images were obtained from far-red and blue channel with the 10× zoom using the Hamamatsu ORCA-ER digital camera. In order to determine the exposure time, wells with or without labelling were used as control. Once the acquisition was complete, images were analysed with the software Cellomics View. Each parameter (Thresholding, Segmentation, Object border) was set manually, to ensure that the automatic cell count reflects the particularity of the cells. The automated fluorescent cell count and nuclei count across 25 fields were averaged to obtain the percentage of fluorescent cells for each transfection condition. The number of fluorescent cells over the number of nuclei was plotted as percentage of fluorescent cells using Graphpad prism software. The experiment was done 3 times and each sample was represented in duplicates.

### Confocal Microscopy

Confocal microscopy was performed with an Olympus FV1000 laser-scanning confocal microscope. Images were sequentially acquired, line-by-line, in order to reduce excitation and emission cross talk, and step size was defined according to the Nyquist-Shannon sampling theorem. Exposure settings that minimized oversaturated pixels in the final images were used. Twelve bit images from each coverslip were then processed with FIJI, and Z-sections were projected on a single plane using maximum intensity under Z-project function and finally converted to 8-bit RGB colour mode. The experiment was repeated 3 times with 3 replicates per condition. At least 3 images were acquired for each coverslip.

### Results

### RT-qPCR

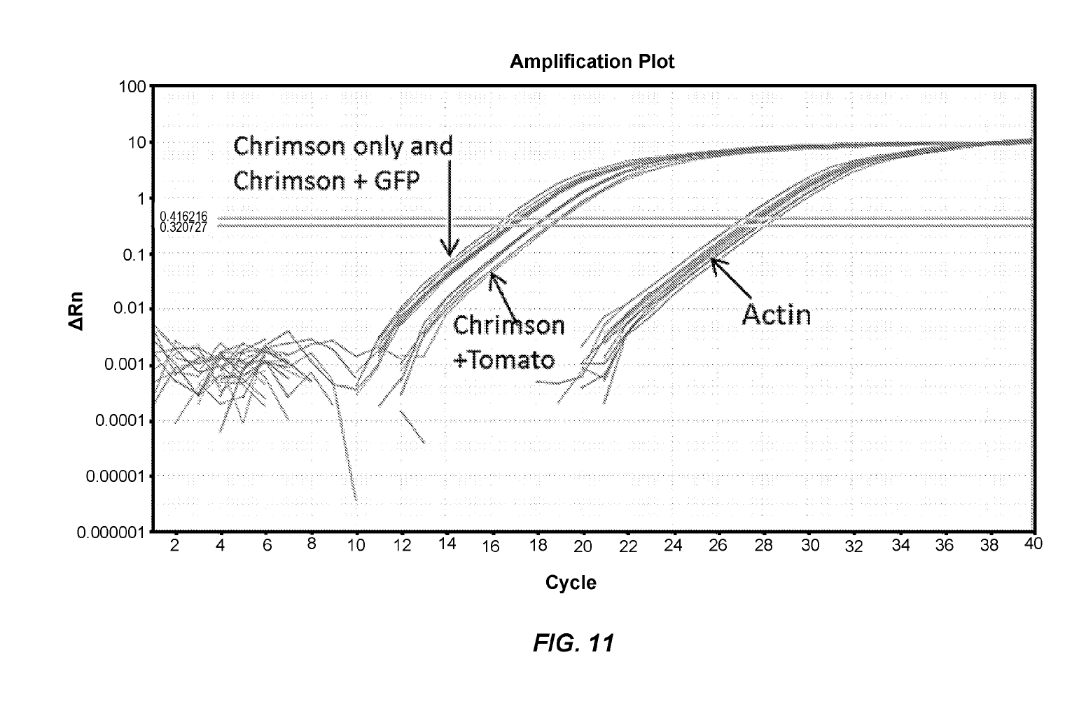

RNA extracted from transfected cells and quantified using RT-qPCR (FIG. 11). Interestingly, we detected higher amounts of ChrimsonR mRNA within the cells transfected with ChrimsonR (480) compared to ChrimsonR-tdTomato (479). Assuming that the transfection was similar between plasmids encoding ChrimsonR and ChrimsonR-tdTomato, this would in principal lead to higher-level expression of ChrimsonR. However, the amount of mRNA present inside the cells does not directly reflect the protein expression levels. Post-translational steps define the overall protein levels and protein localization within the cell. Therefore, in a next set of experiments HEK cells were transfected with either ChrimsonR or ChrimsonR-tdTomato and protein expression was tracked by microscopy.

FIG. 11 shows raw data of RT-PCR for pssAAV-CAG-ChrimsonR-tdTomato, pssAAV-CAG-ChrimsonR and pssAAV-CAG-ChrimsonR-GFP plasmids. Actin gene mRNA expression was similar regardless of construct tested. It appears that the expression of ChrimsonR-tdTomato is slightly lower than the one of ChrimsonR alone and ChrimsonR-GFP.

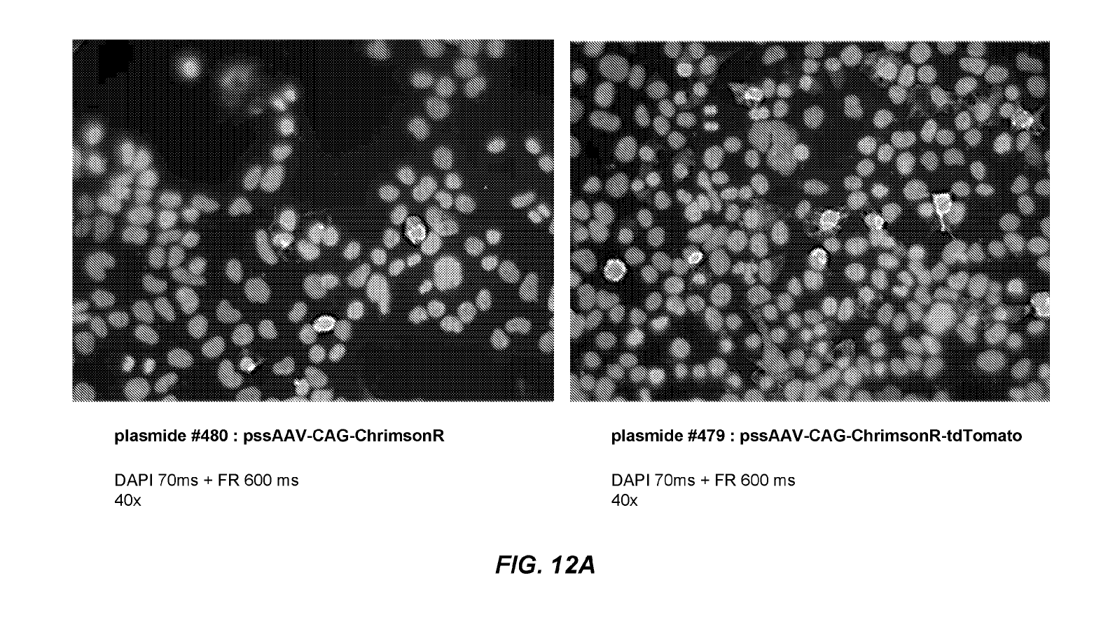

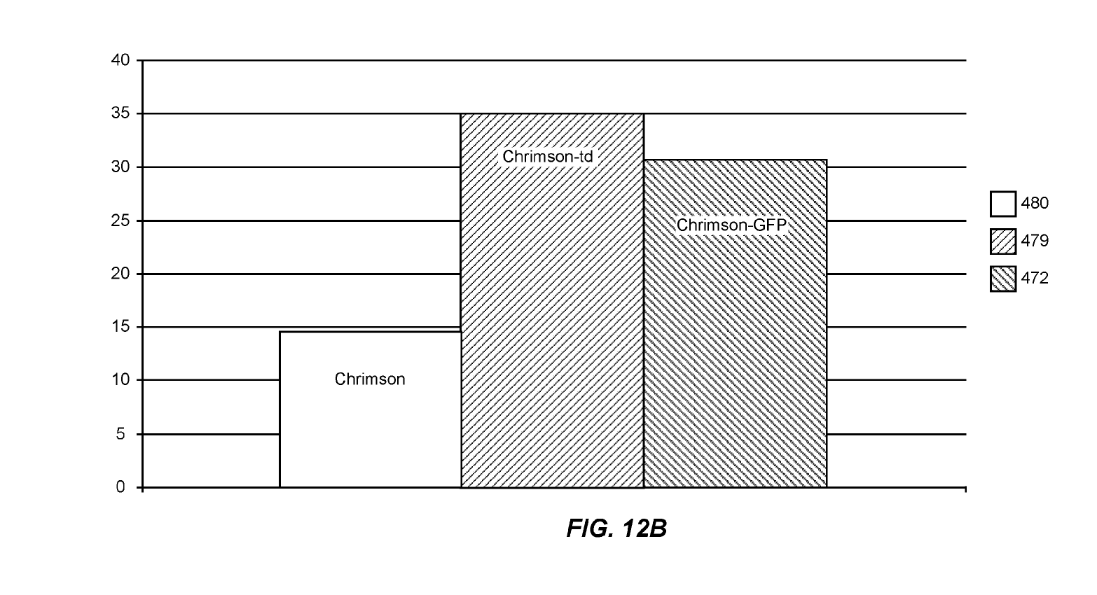

In contrast, the level of ChrimsonR protein was higher when using pssAAV-CAG-ChrimsonR-tdTomato and pssAAV-CAG-ChrimsonR-GFP rather than pssAAV-CAG-ChrimsonR plasmid (FIG. 12). FIG. 12A shows a fluorescence image of HEK293 cells transfected with pssAAV-CAG-ChrimsonR-tdTomato, and pssAAV-CAG-ChrimsonR, respectively. Cell nucleus appear in blue (DAPI staining).

In FIG. 11B, shows that, out of 50,000 analyzed cells, the level of ChrimsonR was higher when ChrimsonR was fused to tdTomato or GFP.

FIG. 12 presents the level of ChrimsonR protein upon transfection of HEK293 cells with pssAAV-CAG-ChrimsonR-tdTomato, pssAAV-CAG-ChrimsonR and pssAAV-CAG-ChrimsonR-GFP plasmids.

### Array Scan Imaging and Quantification

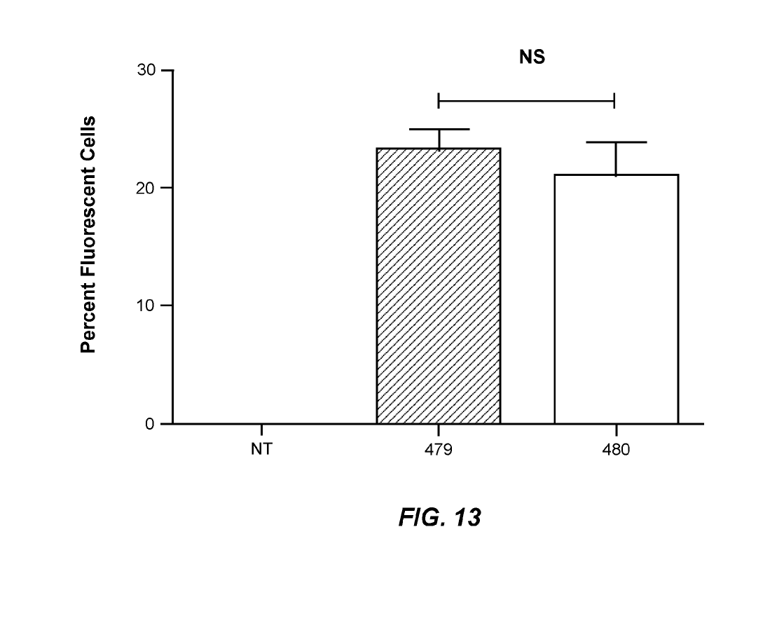

Array scan was used to count the total number of cells (based on their nuclei) as well as the fluorescent cells after anti-ChrimsonR antibody labelling of samples transfected with ChrimsonR (480) versus ChrimsonR-tdTomato (479) plasmid. The difference between the number of cells expressing ChrimsonR fused or not to tdTomato was not significant (FIG. 13). Thus, according to this counting method, a same number of cells was transfected and expressed ChrimsonR regardless of the presence or not of tdTomato. However, the percentage of fluorescent cells does not convey information about the localisation of the fluorescence. Since only ChrimsonR expressed at the membrane can lead to change in membrane potential upon light activation, using confocal microscopy we next investigated the differences in subcellular localisation of ChrimsonR in the presence and absence of tdTomato.

### Confocal Microscopy

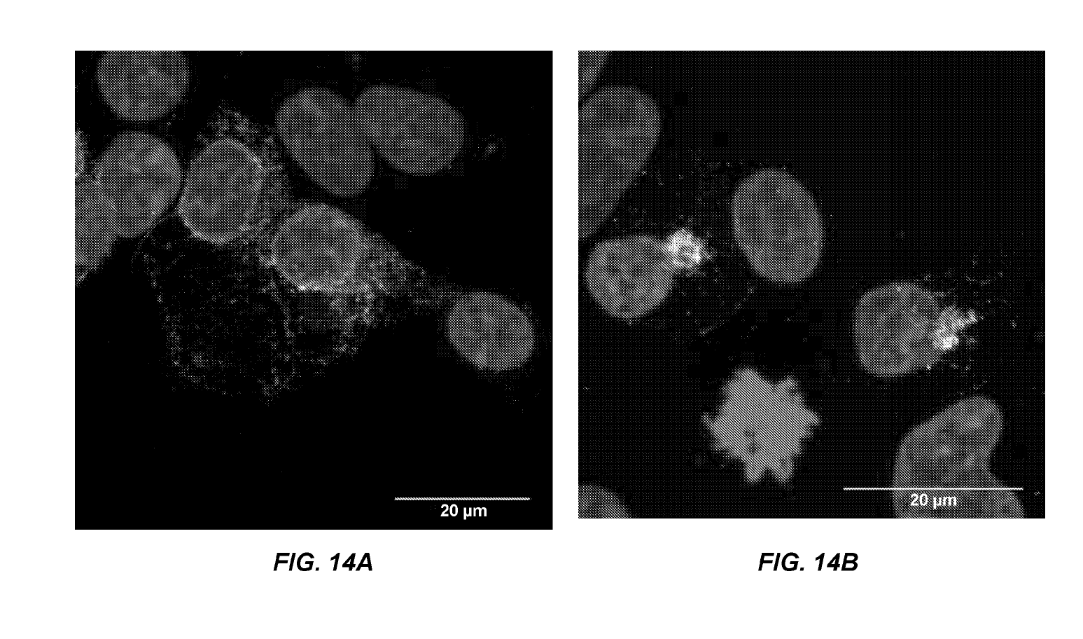

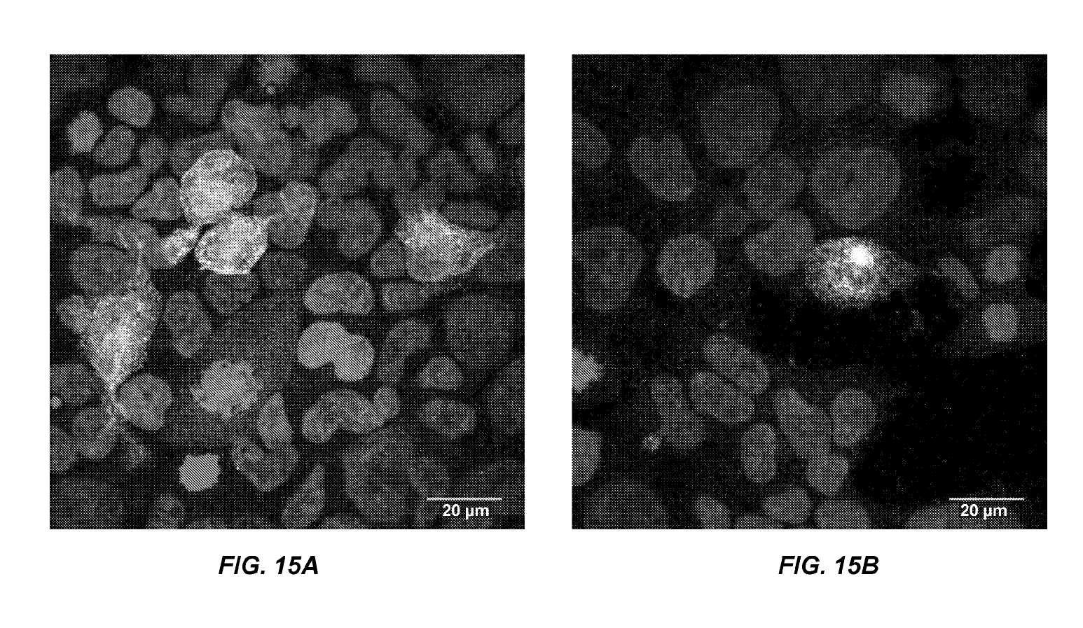

Transfected/infected cells were labelled with antibodies against ChrimsonR and with DAPI as described in Materials and Methods. Coverslips were then mounted and observed with the confocal microscope. Z-stacks acquired using the same parameters were max-projected to obtain representative images of the distribution of ChrimsonR in HEK cells. Our data show that the subcellular localisation of ChrimsonR versus ChrimsonR-tdTomato is significantly different. ChrimsonR remains in the peri-nuclear region in what seems to be the endoplasmic reticulum (FIGS. 14 and 15). ChrimsonR-tdTomato on the other hand, is widely distributed across the cell with no accumulation in peri-nuclear areas (FIGS. 14 and 15). Of note, we did not perform any anti-endoplasmic reticulum staining however staining patterns with ER markers such as KDEL (SEQ ID NO:13) in HEK cells were shown to label a similar area (Wu et al. Biochem J, 464, 13-22, 2014).

### Analysis

Transcription analysis by RT-qPCR indicated that mRNA levels are slightly higher for cells transfected with ChrimsonR expressing plasmid (480) compared to ChrimsonR-tdTomato expressing plasmid (479). However, the percentage of cells expressing ChrimsonR protein in fusion with tdTomato or not was similar after transfection. Confocal microscopic observation of subcellular localization of the optogene showed that ChrimsonR-tdTomato had a different cellular distribution pattern compared to ChrimsonR alone. Whilst ChrimsonR-tdTomato was widely distributed within the cell, ChrimsonR alone essentially accumulated in the endoplasmic reticulum (ER), which might indicate alteration in its release from the ER and subsequent insertion into the membrane. ChrimsonR is a fairly insoluble protein whilst tdTomato is a large and soluble protein (Shaner et al., Nat Methods, 2, 905-909, 2005). Thus, these data suggest that tdTomato might actually improve the solubility of the optogenetic protein and promote the release of ChrimsonR from the ER when it is included as a fusion protein at the C-terminal end of ChrimsonR.

1. A method for reactivating retinal ganglion cells (RGCs) in mammals comprising administering to a mammal a vector expressing an effective amount of Chrimson protein fused to a fluorescent protein.

2. A method of treating or preventing neuron mediated disorders in a subject wherein the method comprises administering to the subject a composition comprising a vector expressing an effective amount of Chrimson protein fused to a fluorescent protein.

3. A method of restoring sensitivity to light in an inner retinal cell wherein the method comprises administering to an inner retinal cell a composition comprising a vector expressing an effective amount of Chrimson protein fused to a fluorescent protein.

4. A method of restoring vision to a subject wherein the method comprises administering to the subject a composition comprising a vector expressing an effective amount of Chrimson protein fused to a fluorescent protein.

5. A method of restoring vision to a subject wherein the method comprises identifying a subject with loss of vision due to a deficit in light perception or sensitivity and administering to the subject a composition comprising a vector expressing an effective amount of Chrimson protein fused to a fluorescent protein.

6. A method of treating or preventing retinal degeneration in a subject wherein the method comprises identifying a subject with retinal degeneration due to loss of photoreceptor function and administering to the subject a composition comprising a vector expressing an effective amount of Chrimson protein fused to a fluorescent protein.

7. A method of restoring photoreceptor function in a human eye wherein the method comprises identifying a subject with loss of vision due to a deficit in light perception or sensitivity and administering to the subject a composition comprising a vector expressing an effective amount of Chrimson protein fused to a fluorescent protein.

8. A method of depolarizing an electrically active cell wherein the method comprises administering to the cell a composition comprising a vector expressing an effective amount of Chrimson protein fused to a fluorescent protein.

9. (canceled)

10. The method of claim 2, wherein the Chrimson protein is Chrimson 88 or Chrimson R.

11. The method of claim 10, wherein the fluorescent protein is selected from Td-Tomato (TdT) protein and green fluorescent protein (GFP).

12. The method of claim 11, wherein the Chrimson protein fused to the tdT protein is more effective in responding to light stimuli compared with Chrimson protein alone.

13. The method of claim 10, wherein the fluorescent protein increases the expression level of the fused Chrimson protein for a given number of cells compared with the expression level of the Chrimson protein alone.

14. The method of claim 13, wherein the expression level of the fused Chrimson protein is increased through enhanced solubility, trafficking, and/or protein conformation of the Chrimson protein.

15. The method of claim 2, wherein the vector is an adenoassociated virus (AAV) vector.

16. The method of claim 15 wherein the AAV vector is selected from AAV2 vector and AAV2.7m8 vector.

17. (canceled)

18. (canceled)

19. The method of claim 2, wherein the vector is injected intravitreally.

20. (canceled)

21. (canceled)

22. (canceled)

23. A composition comprising one or more polynucleotides encoding one or more Chrimson proteins and one or more fluorescent proteins, fused or separately.

24. A method of treating or preventing neuron mediated disorders in a subject, wherein the method comprises administering to the subject the composition of claim 23.

25. A method of reactivating retinal ganglion cells (RGCs) in a subject, treating or preventing neuron mediated disorders in a subject, restoring sensitivity to light in an inner retinal cell in a subject, treating or preventing retinal degeneration in a subject, restoring photoreceptor function in a subject, or depolarizing an electrically active cell in a subject, comprising administering to the subject the composition of claim 23.

26. The method of claim 2, wherein the neuron mediated disorder is a loss of vision due to a deficit in light perception or sensitivity.

27. The method of claim 2, wherein the neuron mediated disorder is a retinal degeneration due to loss of photoreceptor function.

28. The method of claim 26, wherein the Chrimson protein fused to a fluorescent protein is stimulated by light and restores sensitivity to light in an inner retinal cell.

29. The method of claim 28, wherein the inner retinal cell is retinal retinal ganglion cell (RGC) in the subject and, wherein a light stimuli level inducing RGCs response is below radiation safety limit.


SEQUENCE LISTING (13 sequences)

SEQ ID NO: 1 - protein, 350 aa, Artificial Sequence
  source: /note=“Description of Artificial Sequence Synthetic polypeptide”
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 2 - protein, 350 aa, Artificial Sequence
  source: /note=“Description of Artificial Sequence Synthetic polypeptide”
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIRLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 3 - protein, 475 aa, Artificial Sequence
  source: /note=“Description of Artificial Sequence Synthetic polypeptide”
VSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYK

SEQ ID NO: 4 - protein, 238 aa, Artificial Sequence
  source: /note=“Description of Artificial Sequence Synthetic polypeptide”
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 5 - protein, 831 aa, Artificial Sequence
  source: /note=“Description of Artificial Sequence Synthetic polypeptide”
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIRLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTVAAPVVAVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLYGMDELYK

SEQ ID NO: 6 - DNA, 2496 bp, Artificial Sequence
  source: /note=“Description of Artificial Sequence Synthetic polynucleotide”
atggctgagctgatcagcagcgccaccagatctctgtttgccgccggaggcatcaacccttggcctaacccctaccaccacgaggacatgggctgtggaggaatgacacctacaggcgagtgcttcagcaccgagtggtggtgtgacccttcttacggactgagcgacgccggatacggatattgcttcgtggaggccacaggcggctacctggtcgtgggagtggagaagaagcaggcttggctgcacagcagaggcacaccaggagaaaagatcggcgcccaggtctgccagtggattgctttcagcatcgccatcgccctgctgacattctacggcttcagcgcctggaaggccacttgcggttgggaggaggtctacgtctgttgcgtcgaggtgctgttcgtgaccctggagatcttcaaggagttcagcagccccgccacagtgtacctgtctaccggcaaccacgcctattgcctgcgctacttcgagtggctgctgtcttgccccgtgatcctgatcagactgagcaacctgagcggcctgaagaacgactacagcaagcggaccatgggcctgatcgtgtcttgcgtgggaatgatcgtgttcggcatggccgcaggactggctaccgattggctcaagtggctgctgtatatcgtgtcttgcatctacggcggctacatgtacttccaggccgccaagtgctacgtggaagccaaccacagcgtgcctaaaggccattgccgcatggtcgtgaagctgatggcctacgcttacttcgcctcttggggcagctacccaatcctctgggcagtgggaccagaaggactgctgaagctgagcccttacgccaacagcatcggccacagcatctgcgacatcatcgccaaggagttttggaccttcctggcccaccacctgaggatcaagatccacgagcacatcctgatccacggcgacatccggaagaccaccaagatggagatcggaggcgaggaggtggaagtggaagagttcgtggaggaggaggacgaggacacagtggcggcaccggtagtagcagtgagtaagggcgaggaagtgatcaaagagttcatgcggtttaaggtgagaatggaaggaagcatgaacggccacgagttcgaaattgagggagaaggagagggacggccctacgagggcacccagacagccaagctgaaagtgacaaagggcgggcctctgccattcgcttgggacatcctgagcccacagtttatgtacggctccaaggcctatgtgaaacatccagctgacattcccgattataagaaactgagcttccccgaggggtttaagtgggaaagagtgatgaacttcgaggacggaggcctggtgactgtgacccaggacagctccctgcaggatgggaccctgatctacaaggtgaaaatgagagggacaaattttccccctgatggacctgtgatgcagaagaaaactatgggatgggaggcctccaccgaaaggctgtatccacgcgacggggtgctgaaaggagaaatccaccaggctctgaagctgaaagatgggggacattacctggtggagttcaagacaatctacatggccaagaaacctgtgcagctgccaggctactattacgtggacacaaaactggatatcacttcacacaacgaggactacactattgtggagcagtatgaacggagcgaggggagacaccatctgttcctgggccatgggactggaagtaccggctcagggtctagtggaaccgcctcaagcgaggataacaatatggctgtgatcaaagagttcatgaggtttaaggtgcgcatggagggcagcatgaatgggcacgaatttgagattgaaggagagggcgaagggaggccttacgagggcacacagactgccaagctgaaagtgaccaagggaggaccactgcctttcgcttgggatatcctgtctcctcagtttatgtacggaagtaaggcctatgtcaagcatcccgctgacattcctgattacaagaaactgtctttcccagagggctttaagtgggagagagtgatgaattttgaagatggaggcctggtgaccgtgacacaggactcctctctgcaggatggcactctgatctacaaagtcaaaatgcgcggcaccaattttccacccgatgggcccgtgatgcagaagaaaacaatggggtgggaggccagcactgaacggctgtatcctagagacggagtgctgaagggcgaaatccaccaggccctgaagctgaaagacggcggccactacctggtggagttcaaaaccatctacatggccaagaaaccagtgcagctgcccggctattactatgtggacaccaagctggatatcacatcccacaatgaagactacaccattgtggaacagtatgagaggtctgaaggacgccaccatctgtttctgtacggcatggatgagctgtataagtaa

SEQ ID NO: 7 - DNA, 4166 bp, Artificial Sequence
  source: /note=“Description of Artificial Sequence Synthetic polynucleotide”
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctccccccccctccccacccccaattttgtatttatttattttttaattattttgtgcagcgatgggggcggggggggggggggggcgcgcgccaggcggggcggggcggggcgaggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgcatcgatattctgagccgccaccatggctgagctgatcagcagcgccaccagatctctgtttgccgccggaggcatcaacccttggcctaacccctaccaccacgaggacatgggctgtggaggaatgacacctacaggcgagtgcttcagcaccgagtggtggtgtgacccttcttacggactgagcgacgccggatacggatattgcttcgtggaggccacaggcggctacctggtcgtgggagtggagaagaagcaggcttggctgcacagcagaggcacaccaggagaaaagatcggcgcccaggtctgccagtggattgctttcagcatcgccatcgccctgctgacattctacggcttcagcgcctggaaggccacttgcggttgggaggaggtctacgtctgttgcgtcgaggtgctgttcgtgaccctggagatcttcaaggagttcagcagccccgccacagtgtacctgtctaccggcaaccacgcctattgcctgcgctacttcgagtggctgctgtcttgccccgtgatcctgatcagactgagcaacctgagcggcctgaagaacgactacagcaagcggaccatgggcctgatcgtgtcttgcgtgggaatgatcgtgttcggcatggccgcaggactggctaccgattggctcaagtggctgctgtatatcgtgtcttgcatctacggcggctacatgtacttccaggccgccaagtgctacgtggaagccaaccacagcgtgcctaaaggccattgccgcatggtcgtgaagctgatggcctacgcttacttcgcctcttggggcagctacccaatcctctgggcagtgggaccagaaggactgctgaagctgagcccttacgccaacagcatcggccacagcatctgcgacatcatcgccaaggagttttggaccttcctggcccaccacctgaggatcaagatccacgagcacatcctgatccacggcgacatccggaagaccaccaagatggagatcggaggcgaggaggtggaagtggaagagttcgtggaggaggaggacgaggacacagtggcggcaccggtagtagcagtgagtaagggcgaggaagtgatcaaagagttcatgcggtttaaggtgagaatggaaggaagcatgaacggccacgagttcgaaattgagggagaaggagagggacggccctacgagggcacccagacagccaagctgaaagtgacaaagggcgggcctctgccattcgcttgggacatcctgagcccacagtttatgtacggctccaaggcctatgtgaaacatccagctgacattcccgattataagaaactgagcttccccgaggggtttaagtgggaaagagtgatgaacttcgaggacggaggcctggtgactgtgacccaggacagctccctgcaggatgggaccctgatctacaaggtgaaaatgagagggacaaattttccccctgatggacctgtgatgcagaagaaaactatgggatgggaggcctccaccgaaaggctgtatccacgcgacggggtgctgaaaggagaaatccaccaggctctgaagctgaaagatgggggacattacctggtggagttcaagacaatctacatggccaagaaacctgtgcagctgccaggctactattacgtggacacaaaactggatatcacttcacacaacgaggactacactattgtggagcagtatgaacggagcgaggggagacaccatctgttcctgggccatgggactggaagtaccggctcagggtctagtggaaccgcctcaagcgaggataacaatatggctgtgatcaaagagttcatgaggtttaaggtgcgcatggagggcagcatgaatgggcacgaatttgagattgaaggagagggcgaagggaggccttacgagggcacacagactgccaagctgaaagtgaccaagggaggaccactgcctttcgcttgggatatcctgtctcctcagtttatgtacggaagtaaggcctatgtcaagcatcccgctgacattcctgattacaagaaactgtctttcccagagggctttaagtgggagagagtgatgaattttgaagatggaggcctggtgaccgtgacacaggactcctctctgcaggatggcactctgatctacaaagtcaaaatgcgcggcaccaattttccacccgatgggcccgtgatgcagaagaaaacaatggggtgggaggccagcactgaacggctgtatcctagagacggagtgctgaagggcgaaatccaccaggccctgaagctgaaagacggcggccactacctggtggagttcaaaaccatctacatggccaagaaaccagtgcagctgcccggctattactatgtggacaccaagctggatatcacatcccacaatgaagactacaccattgtggaacagtatgagaggtctgaaggacgccaccatctgtttctgtacggcatggatgagctgtataagtaaagaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 8 - DNA, 783 bp, Artificial Sequence
  source: /note=“Description of Artificial Sequence Synthetic polynucleotide”
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctccccccccctccccacccccaattttgtatttatttattttttaattattttgtgcagcgatgggggcggggggggggggggggcgcgcgccaggcggggcggggcggggcgaggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgc

SEQ ID NO: 9 - DNA, 20 bp, Artificial Sequence
  source: /note=“Description of Artificial Sequence Synthetic primer”
gctcttttccagccttcctt

SEQ ID NO: 10 - DNA, 20 bp, Artificial Sequence
  source: /note=“Description of Artificial Sequence Synthetic primer”
cttctgcatcctgtcagcaa

SEQ ID NO: 11 - DNA, 20 bp, Artificial Sequence
  source: /note=“Description of Artificial Sequence Synthetic primer”
acacctacaggcgagtgctt

SEQ ID NO: 12 - DNA, 20 bp, Artificial Sequence
  source: /note=“Description of Artificial Sequence Synthetic primer”
tccgtaagaagggtcacacc

SEQ ID NO: 13 - protein, 4 aa, Artificial Sequence
  source: /note=“Description of Artificial Sequence Synthetic peptide”
KDEL